(19) United States
(12) Patent Application Publication
(10) Pub. No.: US 2025/0228780 A1
(43) Pub. Date: Jul. 17, 2025

(54) NOVEL COMPOSITION

(71) Applicant: Debiopharm International S.A., Lausanne (CH)

(72) Inventors: Benoît Plagès, La Tour-De-Peilz (CH); Antoine Touzet, Vevey (CH); Anne Bellon, Pully (CH); Laurence Fajas Descamps, Mollie Margot (CH)

(73) Assignee: Debiopharm International S.A., Lausanne (CH)

(21) Appl. No.: 18/853,800

(22) PCT Filed: Mar. 31, 2023
(86) PCT No.: PCT/EP2023/058510

§ 371 Date: Oct. 3, 2024

(30) Foreign Application Priority Data
Apr. 4, 2022 (EP) 22166467.5
Sep. 7, 2022 (EP) 22194434.1

(51) Int. Cl. A61K 9/16; A61K 9/00; A61K 38/31; A61K 41/10

(52) U.S. Cl. CPC A61K 9/1647; A61K 9/0024; A61K 9/1694; A61K 38/31; A61K 41/10

(57) ABSTRACT

A pharmaceutical composition comprising biodegradable polymer microparticles comprising a somatostatin analogue or a pharmaceutically acceptable salt thereof, wherein said pharmaceutical composition preferably provides a sustained release of the somatostatin analogue, or a pharmaceutically acceptable salt thereof, over 30 days or more.

### FIELD OF THE INVENTION

The present invention relates to a pharmaceutical composition comprising biodegradable polymer microparticles comprising a somatostatin analogue or a pharmaceutically acceptable salt thereof. The invention further relates to said pharmaceutical composition for use as a medicament.

### BACKGROUND

Somatostatin analogues e.g., octreotide, lanreotide, and pasireotide, are peptide drugs that can be used in a variety of therapeutic applications e.g., in the treatment of acromegaly, carcinoid syndrome associated with neuroendocrine tumours, and vasoactive intestinal peptide tumours (VIPoma tumours). Often long term treatment is required with these peptide drugs, and ordinarily they are administered parenterally e.g., via injection, which can have drawbacks, especially when repeated and frequent administration e.g., daily or weekly, is required over long time frames.

In view of the drawbacks associated with frequent parenteral administration, pharmaceutical manufacturers have focused on developing sustained release compositions that can serve to reduce the number of administrations needed over the long term. Such sustained release pharmaceutical compositions are frequently poly-lactic-co-glycolic acid copolymer (PLGA) based. However, formulating PLGA sustained release pharmaceutical compositions to have acceptable somatostatin analogue release profiles is challenging, especially if the desired sustained release profile is more than 1 month (30 days) e.g., 2 months (60 days) or more, or even 3 months (90 days) or more. A particular challenge in such cases is avoiding an unacceptably high initial release (burst) of the somatostatin analogue following administration, whilst at the same time ensuring a sufficient and stable release of it over the entire sustained release period.

In response to the aforementioned challenges formulators have developed sustained release pharmaceutical compositions comprising blends of microparticles made from different PLGAs.

However, such compositions can be complex and expensive to produce. Also, depending on the desired sustained release period, high doses of the somatostatin analogue (and in consequence high amounts of PLGA microparticles) may need to be administered, which may result in a formulation with syringeability and injectability issues.

Accordingly, there is still a need for new sustained release pharmaceutical compositions that can provide a sustained release of a somatostatin analogue, especially over longer periods of time e.g., more than 1 month e.g., 2 months or more, 3 months or more, or even longer. In particular there is a need for such somatostatin sustained release compositions that have acceptable somatostatin release profiles e.g., sufficient release of the somatostatin analogue over the entire desired sustained release period, and an acceptable burst (which may minimise, or minimise the risk of, side effects/adverse reactions associated with the administration of the somatostatin analogue). Further, there is a need for such pharmaceutical compositions that are not complex or expensive to manufacture, that are convenient and easy to use, that have an acceptable shelf life, that are safe and well tolerated by patents, and that minimises patient discomfort e.g., caused by the repeated administrations associated with long term treatment, and/or syringeability and injectability issues.

It is the object of the following invention to address one or more of the aforementioned needs.

The inventors have now surprisingly found that a pharmaceutical composition as described herein can fulfill one or more of the aforementioned needs. In particular the inventors have found that a pharmaceutical composition in accordance with the invention may release a somatostatin analogue over 30 days or more e.g., 60 days or more, 90 days or more, or even longer, and may have an acceptable burst. Further, the inventors have also surprisingly found that the bioavailability of a somatostatin analogue comprised within the pharmaceutical composition of the invention may be surprisingly high in comparison to currently marketed somatostatin analogue e.g., octreotide, sustained release formulations e.g., Sandostatin® LAR®, which may enable reduced doses of a somatostatin analogue (and therefore lower amounts of PLGA microparticles) to be administered whilst achieving the same effect as higher doses in other formulations.

### SUMMARY OF THE INVENTION

1. A pharmaceutical composition comprising biodegradable polymer microparticles comprising a somatostatin analogue, or a pharmaceutically acceptable salt thereof, wherein said biodegradable polymer comprises PLGA having:
   a molar ratio of lactide to glycolide of 80:20 to 90:10, and
   an inherent viscosity of 0.2 to 0.4 dl/g as measured at a concentration of 0.5 w/w % in chloroform at 25° C.,
and wherein said biodegradable polymer microparticles have:
   a drug loading of 10 to 15 w/w %,
   a Dv50 of 30 μm to 90 μm, and
   a specific surface area of less than 0.50 m²/g, and preferably less than 0.40 m²/g, as measured by gas adsorption,
and wherein preferably said pharmaceutical composition provides a sustained release of the somatostatin analogue, or a pharmaceutically acceptable salt thereof, over 30 days or more, and more preferably over 30 days to 200 days.
2. The pharmaceutical composition according to item 1 wherein said pharmaceutical composition provides a sustained release of the somatostatin analogue over 60 days or more, and preferably 60 to 200 days.
3. The pharmaceutical composition according to item 1 wherein said pharmaceutical composition provides a sustained release of the somatostatin analogue over 90 days or more, and preferably 90 to 200 days.
4. The pharmaceutical composition according to any preceding item wherein the PLGA defined in item 1 has a molar ratio of lactide to glycolide of 83:17 to 87:13, and an inherent viscosity of 0.25 to 0.35 dl/g as measured at a concentration of 0.5 w/w % in chloroform at 25° C.
5. The pharmaceutical composition according to any preceding item wherein the PLGA defined in any preceding item comprises less than 0.5 w/w % of residual lactide and/or glycolide monomer.
6. The pharmaceutical composition according to any preceding item wherein the biodegradable polymer microparticles defined in any preceding item have a drug loading of 11 to 14 w/w %, and preferably 11.5 to 12.5 w/w %.
7. The pharmaceutical composition according to any preceding item wherein the biodegradable polymer microparticles defined in any preceding item are biodegradable polymer microspheres.
8. The pharmaceutical composition according to any preceding item wherein, the biodegradable polymer microparticles defined in any preceding item have:
   a Dv50 of 50 μm to 80 μm, preferably 60 μm to 75 μm, and/or
   a specific surface area of 0.05 to 0.3 m²/g as measured by gas adsorption.
9. The pharmaceutical composition according to any preceding item wherein the somatostatin analogue is octreotide or a pharmaceutically acceptable salt thereof, and preferably wherein the somatostatin analogue is a pharmaceutically acceptable salt of octreotide selected from the group consisting of octreotide acetate and octreotide pamoate.
10. The pharmaceutical composition according to any preceding item wherein the biodegradable polymer defined in any preceding item comprises at least 50 w/w % of the PLGA defined in any preceding item, preferably at least 85 w/w %, and more preferably 100 w/w %.
11. The pharmaceutical composition according to any preceding item wherein the biodegradable polymer microparticles defined in any preceding item make up at least 50 w/w % of the total of all biodegradable polymer microparticles comprised in the pharmaceutical composition, preferably at least 75 w/w %, and more preferably at least 80 w/w %.
12. The pharmaceutical composition according to any preceding claim wherein the composition is characterized by a release of the somatostatin analogue that fulfils the following criteria: release of less than 7% of the somatostatin analogue comprised therein over 5 hours, wherein said release is measured in vitro (at 37° C. in 900 mL of a pH4 100 mM acetate buffer) according to the method described in the European Pharmacopoeia 10, 2.9.3., wherein said composition is tested in an amount equating to 30 mg of the somatostatin analogue, and wherein said somatostatin analogue is preferably octreotide.
13. The pharmaceutical composition according to any preceding claim wherein the composition is characterised by a release of the somatostatin analogue that fulfils the following test criteria: release of less than 3% of the somatostatin analogue comprised therein over 5 hours, wherein said release is measured in vitro (at 37° C. in 900 mL of a pH4 100 mM acetate buffer) according to the method described in the European Pharmacopoeia 10, 2.9.3., wherein said composition is tested in an amount equating to 30 mg of the somatostatin analogue, and wherein said somatostatin analogue is preferably octreotide.
14. The pharmaceutical composition according to any preceding item wherein the pharmaceutical composition is a liquid suspension comprising biodegradable polymer microparticles or is a composition of dried biodegradable polymer microparticles.
15. The pharmaceutical composition according to item 14 wherein the pharmaceutical composition is a liquid suspension comprising biodegradable polymer microparticles and wherein the biodegradable polymer microparticles are suspended in a water-vehicle or a non-aqueous vehicle, and preferably wherein the non-aqueous liquid vehicle is a pharmaceutically acceptable oil essentially consisting of one or more medium chain triglycerides.
16. The pharmaceutical composition according to item 15 wherein the liquid comprises the microparticles in a concentration of 100 mg/mL to 500 mg/mL, preferably, 100 mg/mL to 375 mg/ml, and more preferably 125 mg/mL to 375 mg/mL.
17. The pharmaceutical composition according to any preceding item wherein said pharmaceutical composition is sterilised by irradiation.
18. A pharmaceutical composition as defined in any preceding item for use as a medicament.
19. A pharmaceutical composition as defined in any of items 1 to 17 for use in the treatment of a disease in which a somatostatin analogue, or a pharmaceutically acceptable salt thereof, has a therapeutic effect, and wherein preferably the disease is selected from the group consisting of autosomal dominant polycystic kidney disease, Cushing's disease, polycystic liver disease, acromegaly, gigantism, TSH-secreting pituitary adenomas, carcinoid syndrome, vasoactive intestinal peptide tumours, and neuroendocrine neoplasms including neuroendocrine tumours including gastro-entero-pancreatic neuroendocrine tumours, and preferably is selected from acromegaly and gastro-entero-pancreatic neuroendocrine tumours.
20. The pharmaceutical composition as defined in any of items 1 to 17 for use according to item 18 or 19 wherein the composition provides a sustained release of a somatostatin analogue over 60 days or more and is to be administered once about every 60 days in a dose corresponding to a dose of octreotide of 25 mg to 105 mg, or the equivalent dose of a pharmaceutically acceptable salt thereof, and preferably wherein the composition provides a sustained release of octreotide, or a pharmaceutically acceptable salt thereof, over 84 days or more and is administered once every 84 days in a dose corresponding to a dose of octreotide of 60 mg, 90 mg, or 120 mg, or the equivalent dose of a pharmaceutically acceptable salt thereof.
21. The pharmaceutical composition as defined in any of items 1 to 17 for use according to item 18 or 19 wherein the composition provides a sustained release of a somatostatin analogue over 90 days or more and is to be administered once about every 90 days in a dose corresponding to a dose of octreotide of 25 mg to 105 mg, or the equivalent dose of a pharmaceutically acceptable salt thereof.
22. The pharmaceutical composition for use according to any one of items 18 to 21 wherein the pharmaceutical composition is to be administered to a patient parenterally and preferably via intra-muscular or subcutaneous injection, wherein the subcutaneous injection is preferably deep-subcutaneous.
23. A kit comprising:
   i. the pharmaceutical composition as defined in any one of items 1 to 17,
   ii. optionally a vehicle for reconstitution, and
   iii. a vial or a syringe optionally prefilled with the pharmaceutical composition of item (i).
24. A method of manufacturing a biodegradable polymer microparticle defined in claim any preceding item wherein said biodegradable polymer comprises at least 50 w/w %, preferably 85 ww % and most preferably 100 w/w %, of the PLGA defined in any preceding item and wherein said method comprises the steps of:
   i. Preparing an organic phase comprising an organic solvent mix, preferably a mix of dichloromethane and methanol in a weight ratio of 80:20 to 95:5, the biodegradable polymer in a concentration of from 10 w/w % to 30 w/w %, and the somatostatin analogue or pharmaceutically acceptable salt thereof in a concentration adequate to achieve a predetermined drug loading of 10 w/w % to 15 w/w %,
   ii. Preparing an aqueous phase comprising water, 0.1 w/v % to 10 w/v % of a stabiliser, preferably PVA, and 2 w/w % to 5 w/w % of a salting-out agent, preferably sodium chloride,
   iii. Continuously mixing the organic and aqueous phases of steps i. and ii. in a volume ratio of 1:80 to 1:250, to form an emulsion,
   iv. Removing the organic solvents by solvent evaporation or solvent extraction from the emulsion of step iii,
   v. Drying the biodegradable polymer microparticles obtained in step iv and sieving them through an appropriately sized sieve,
   vi. optionally repeating step v until any residual organic solvent is below a predetermined level,
      wherein, with the exception of the drug loading, all w/w % and w/v % in the above process are based on the weight or volume of the respective solution.

### BRIEF DESCRIPTION OF THE FIGURES

FIG. 1 In vivo pharmacokinetic release profile of a suspension of composition D microspheres (Example 4).

FIG. 2 In vivo pharmacokinetic release profile of a suspension of composition A microspheres (Example 5).

FIG. 3 In vivo pharmacokinetic release profile of aqueous and non-aqueous suspensions of composition D microparticles by route of administration and reconstitution vehicle (Example 9).

FIG. 4 Mean plasma octreotide levels over the investigational period resulting from a single administration of composition D microparticles (90 mg of octreotide), or 3×30 mg (1 per month) administrations of Sandostatin LAR (Example 10B).

FIG. 5 Change in IGF1 serum levels over the investigational period resulting from a single administration of composition D microparticles (90 mg of octreotide), or 3×30 mg (1 per month) administrations of Sandostatin LAR (Example 10B).

### DETAILED DESCRIPTION

In a first aspect of the present invention there is provided a pharmaceutical composition comprising biodegradable polymer microparticles comprising a somatostatin analogue or a pharmaceutically acceptable salt thereof, wherein said biodegradable polymer comprises PLGA having:
a molar ratio of lactide to glycolide of 80:20 to 90:10, and
an inherent viscosity of 0.2 to 0.4 dl/g as measured at a concentration of 0.5 w/w % in chloroform at 25° C.,
   and wherein, said biodegradable polymer microparticles have:
a drug loading of 10 to 15 w/w %,
a Dv50 of 30 μm to 90 μm, and
an average specific surface area of less than 0.5 m²/g, and preferably less than 0.4 m²/g, as measured by gas adsorption.

The term “biodegradable polymer” as used herein refers to polymers or mixtures of polymers that are degraded in response to contact with bodily fluid e.g., after injection into a patient. Non limiting examples of biodegradable polymers include polylactic acids, poloxamer, polyglycolic acids, polycaprolactones, polycarbonates, polyesteramides, polyanhydrides, polyamino acids, polyethyleneglycol (PEG), polyorthoesters, polycyanoacrylates (and copolymers), PLGA, and mixtures of any of the foregoing, as well as copolymers based on two or more repeating units forming the polymers listed above. In the context of the present invention, preferred biodegradable polymers are polylactic acids, polyglycolic acids, PEG, PLGA, and mixtures of any of the foregoing, as well as copolymers based on two or more repeating units forming the preferred polymers listed. For instance, the biodegradable polymer may comprise PLGA in a mixture with any other biodegradable polymer and may comprise especially PLGA in a mixture with any of the biodegradable polymers other than PLGA that are listed above.

The term “biodegradable polymer microparticle” as used herein refers to a monolithic microparticle formed from one or more biodegradable polymer. Said microparticles have an internal continuous biodegradable polymer matrix. Said microparticles may be of any shape, including spherical or irregular. The term encompasses microspheres and microgranules.

The biodegradable polymer used to form the biodegradable polymer microparticles comprised in the pharmaceutical composition of the invention comprises PLGA having a molar ratio of lactide to glycolide of 80:20 to 90:10, and an inherent viscosity of 0.2 to 0.4 dl/g as measured at a concentration of 0.5 w/w % in chloroform at 25° C.

In an embodiment, said PLGA has a molar ratio of lactide to glycolide of 82:18 to 88:12 e.g., 83:17 to 87:13.

In an embodiment, said PLGA has an inherent viscosity of 0.2 to 0.4 dl/g as measured at a concentration of 0.5 w/w % in chloroform at 25° C.

In a more specific embodiment said PLGA has a molar ratio of lactide to glycolide of 83:17 to 87:13 and an inherent viscosity of 0.25 to 0.35 dl/g as measured at a concentration of 0.5 w/w % in chloroform at 25° C.

Said PLGA may be linear or branched, hyperbranched, comb-like branched, dendrimer-like branched, T-shaped, star-shaped, or any mixture thereof. In an embodiment of the invention said PLGA is linear PLGA.

Said PLGA may be poly D, L-lactide-co-glycolide copolymer.

Said PLGA may comprise less than 5 w/w % e.g., less than 3 w/w %, of residual lactide and/or glycolide monomer.

In an embodiment said PLGA comprises less than 0.5 w/w % of residual lactide and/or glycolide monomer. Most preferably said PLGA is free from residual lactide and/or glycolide monomer i.e., comprises 0% residual lactide and/or glycolide monomer.

The end groups of said PLGA are not limited. Example of possible end groups include hydroxy, ester e.g., a stearyl group, acidic e.g., a carboxy group, or the like.

In an embodiment of the invention the end groups of said PLGA are acidic and preferably are carboxy groups.

The molar ratio of the lactide to glycolide of PLGA can be measured by using conventional methods, for example by Nuclear Magnetic Resonance (¹H NMR).

In the present invention the inherent viscosity of PLGA is measured at a concentration of 0.5 w/w % in chloroform at 25° C. Said inherent viscosity may be measured using conventional methods e.g., The Capillary Viscometer Method as described in “European Pharmacopoeia”, 10.0, chapter 2.2.9.

As understood by the skilled person, the inherent viscosity and molar ratio of lactide to glycolide of PLGA are an average over a certain range e.g., indicated in the specifications of the manufacturers of PLGA.

Any PLGA having the required attributes e.g., molar ratio of lactide to glycolide and inherent viscosity, as set out herein, may be comprised in the biodegradable polymer defined herein i.e the biodegradable polymer used to form the biodegradable polymer microparticles defined herein and comprised in the pharmaceutical composition of the invention.

Non limiting examples of commercially available PLGA include RESOMER® by Evonik, LACTEL® (by Evonik), MEDISORB® by Evonik, PURASORB® by Corbion, and Expansorb® by Seqens.

The biodegradable polymer microparticles comprised in the sustained release pharmaceutical composition of the invention comprise a somatostatin analogue, or pharmaceutically acceptable salt thereof. Said somatostatin analogue, or pharmaceutically acceptable salt thereof, may be loaded in the biodegradable polymer microparticles in a drug loading of 10 to 15 w/w %.

The term “drug loading” as used herein refers to the weight ratio (presented as a percentage) of the somatostatin analogue mass to the combined total mass of the biodegradable polymer microparticles i.e., as expressed by the following equation:

$$Weight ratio = m ( S A ) / T ( B P M )$$

with m(SA) representing the weight of the somatostatin analogue and T(BPM) representing the weight of biodegradable polymer microparticles.

In the case of using a pharmaceutically acceptable salt of a somatostatin analogue, the weight ratio is calculated on the basis of the weight of a molar amount of free base of the somatostatin analogue, which is the same molar amount as the pharmaceutically acceptable salt of a somatostatin analogue comprised in the biodegradable polymer microparticles.

Drug loading can be measured using conventional methods for example using high performance liquid chromatography (HPLC) e.g., an appropriate weight e.g., 20 mg, of the biodegradable polymer microparticles can be weighed, dissolved in dimethylsufoxide (DMSO), and the sample can be analysed by HPLC assay e.g., according to a method set out in the European pharmacopoeia e.g., in the octreotide general monograph for octreotide.

In an embodiment of the invention the biodegradable polymer microparticles comprised in the pharmaceutical composition of the invention have a drug loading of 11 w/w % to 14 w/w % e.g., 11 w/w % to 13 w/w %, 11.5 w/w % to 13 w/w %, 12 w/w % to 13 w/w %. In a more specific embodiment, the biodegradable polymer microparticles have a drug loading of 11.5 to 13%, e.g., 12 to 13 w/w %, and in an even more specific embodiment 11.5 w/w % to 12.5 w/w % e.g., 12 w/w % to 12.5 w/w %.

The term “biodegradable polymer microgranules” as used herein refers to non-spherical irregularly shaped monolithic microparticles formed from one or more biodegradable polymer. Said microgranules have an internal continuous biodegradable polymer e.g., PLGA, matrix.

The term “biodegradable polymer microspheres” as used herein refers to spherical or substantially spherical regularly shaped monolithic microparticles formed from one or more biodegradable polymer. Said microspheres have an internal continuous biodegradable polymer e.g., PLGA, matrix.

Biodegradable polymer microgranules may for instance be made by a dry process e.g., extrusion and grinding, and biodegradable polymer microspheres may for instance be made by a wet process e.g., emulsion and precipitation.

In a specific embodiment of the invention the biodegradable polymer microparticles are biodegradable polymer microspheres.

The biodegradable polymer microparticles e.g., biodegradable polymer microspheres, comprised in the pharmaceutical composition of the invention may have a median particle size i.e., Dv50, of 30 μm to 90 μm e.g., 30 μm to 80 μm, 40 μm to 90 μm, 40 μm to 80 μm, 50 μm to 90 μm, 60 μm to 80 μm e.g., 60 μm to 70 μm, 60 μm to 65 μm.

Said biodegradable polymer microparticles may also additionally have one or more of the following particle size values:
a Dv90 of 70 μm to 200 μm and preferably 100 μm to 150 μm e.g., 100 μm to 140 μm, 100 μm to 130 μm, 100 μm to 125 μm,
a Dv10 of 5 μm to 50 μm and preferably 20 μm to 40 μm e.g., 20 μm to 30 μm, 20 μm to m.

In a specific embodiment said biodegradable polymer microparticles comprised in the composition of the invention have a Dv50 of 60 μm to 80 μm e.g., 60 μm to 75 μm e.g., 60 μm to 70 μm e.g., 62 μm to 65 μm, a Dv90 of 80 μm to 200 μm and preferably 100 μm to 150 μm e.g., 100 μm to 140 μm, 100 μm to 130 μm, 100 μm to 125 μm, and a Dv10 of 5 μm to 50 μm and preferably 20 μm to 40 μm e.g., 20 μm to 30 μm, 20 μm to 25 μm.

The DvX means that X % of the volume of the biodegradable polymer microparticles sample is below the indicated diameter value e.g., a Dv50 of 90 μm means that there is 50% of the volume of the biodegradable polymer microparticles sample below 90 μm in diameter e.g. 20 μm, 30 μm, 40 μm, 50 μm, 60 μm, 70 μm, 80 μm in diameter. The DvX e.g., Dv50, is determined by wet laser diffraction e.g., as described in the European pharmacopoeia V.10.8 chapter 2.9.31. The DvX may, for example, be determined as described in Example 3. The particle size measurement may be conducted by wet laser diffraction e.g., using a Malvern Mastersizer 3000 equipped with a Hydro Medium Volume (MV) dispersion unit. The biodegradable polymer microparticles may be suspended (at room temperature e.g., 20-25° C.) in an aqueous based medium comprising a surfactant e.g., polysorbate 80 (0.1%), the sample may then be dispersed in purified water in a hydro MV dispersion unit (Stirring speed 2000 rpm) until reaching an obscuration between 10% and 20%, following this the sample undergoes ultra-sonification for 3 mins (hydro MV dispersion unit setting medium (50%)), the sample is then analysed using a Malvern mastersizer 3000. Results are calculated based on the Mie theory (real particle refractive index of 1.52 and imaginary particle refractive index of 0.001). The analysis model general purpose may be used.

In an embodiment said biodegradable polymer microparticles have a Dv50 of 50 μm to 80 μm, and more specifically 60 μm to 80 μm.

The biodegradable polymer microparticles defined herein and comprised in the pharmaceutical composition of the invention may have an average specific surface area of less than 0.5 m²/g e.g., 0.49 m²/g, 0.48 m²/g, 0.47 m²/g, 0.46 m²/g, 0.45 m²/g or less, e.g., 0.44 m²/g, 0.43 m²/g, 0.42 m²/g, 0.41 m²/g, 0.4 m²/g e.g., less than 0.4 m²/g e.g., 0.39 m²/g, 0.38 m²/g, 0.37 m²/g, 0.36 m²/g, 0.35 m²/g, 0.34 m²/g, 0.33 m²/g, 0.32 m²/g, 0.31 m²/g, 0.3 or less e.g., 0.29 m²/g, 0.28 m²/g, 0.27 m²/g, 0.26 m²/g, 0.25 m²/g. The specific surface area may, for example be 0.05 to less than 0.5 m²/g e.g., 0.05 to 0.45 m²/g, 0.05 to 0.4 m²/g or less, 0.05 to 0.35 m²/g or less, 0.05 to 0.3 m²/g or less, 0.05 to 0.25 m²/g or less, wherein the specific surface area is determined by gas e.g., nitrogen or krypton gas, adsorption e.g., using a N₂-BET or Kr-BET adsorption method as described in the European pharmacopoeia 10.8 chapter 2.9.26., for instance as described in Example 3.

The specific surface area refers to the total surface area per unit mass of the biodegradable polymer microparticles defined herein and comprised in the composition of the invention. The specific surface area value may be an average calculated over multiple batches of the biodegradable polymer microparticles e.g., 2, 3, 4, 5, or 6 batches. In an embodiment the specific surface area is determined by nitrogen adsorption using the Brunauer-Emmett-Teller (N2-BET) theory, for instance as described in Example 3.

In an embodiment the biodegradable polymer microparticles defined herein have a specific surface area of 0.05 to 0.3 m²/g, and more specifically 0.1 to 0.28 m²/g.

In a more specific embodiment said biodegradable polymer microparticles comprised in the composition of the invention have a Dv50 of 50 μm to 80 μm e.g., 60 μm to 75 μm e.g., 60 μm to 70 μm e.g., 62 μm to 65 μm, and an average specific surface area of 0.05 to 0.3 m²/g e.g., 0.1 to 0.28 m²/g, 0.1 to 0.25 m²/g, 0.20 to 0.21 m²/g, e.g., 0.15 m²/g, 0.2 m²/g, 0.21 m²/g, 0.206 m²/g, 0.25 m²/g, wherein the specific surface area is measured by gas e.g., nitrogen or krypton gas adsorption e.g., as determined using the N2-BET or Kr-BET adsorption method, for instance as described in Example 3.

In an even more specific embodiment said biodegradable polymer microparticles comprised in the composition of the invention have a Dv50 of 60 μm to 80 μm e.g., 60 μm to 75 μm e.g., 60 μm to 70 μm e.g., 62 μm to 65 μm, a Dv90 of 80 μm to 200 μm and preferably 100 μm to 150 μm e.g., 100 μm to 140 μm, 100 μm to 130 μm, 100 μm to 125 μm, and a Dv10 of 5 μm to 50 μm and preferably 20 μm to 40 μm e.g., 20 μm to 30 μm, 20 μm to 25 μm, and an average specific surface area of 0.05 to 0.3 m²/g e.g., 0.1 to 0.28 m²/g, wherein the specific surface area is measured by gas e.g., nitrogen or krypton gas adsorption e.g., as determined using the N2-BET or Kr-BET adsorption method, for instance as described in Example 3.

In another embodiment the biodegradable polymer microparticles comprised in the composition of the invention have:
PLGA having a molar ratio of lactide to glycolide of 83:17 to 87:13,
an inherent viscosity of 0.25 to 0.35 dl/g as measured at a concentration of 0.5 w/w % in chloroform at 25° C.
a Dv50 of 60 μm to 75 μm e.g., 60 μm to 70 μm e.g., 62 μm to 65 μm,
an average specific surface area of 0.05 to 0.3 m²/g e.g., 0.1 to 0.28 m²/g, 0.1 to 0.25 m²/g, 0.20 to 0.21 m²/g, e.g., 0.15 m²/g, 0.2 m²/g, 0.21 m²/g, 0.206 m²/g, 0.25 m²/g, wherein the specific surface area is measured by gas e.g., nitrogen or krypton gas adsorption e.g., as determined using the N2-BET or Kr-BET adsorption method, for instance as described in Example 3, and
a drug loading of 11.5 to 13%, e.g., 11.5 w/w % to 12.5 w/w % e.g., 12 w/w % to 12.5 w/w %.

In another embodiment the biodegradable polymer microparticles comprised in the composition of the invention have:
PLGA having a molar ratio of lactide to glycolide of 83:17 to 87:13,
an inherent viscosity of 0.25 to 0.35 dl/g as measured at a concentration of 0.5 w/w % in chloroform at 25° C.
a Dv50 of 60 μm to 75 μm e.g., 60 μm to 70 μm e.g., 62 μm to 65 μm,
a Dv90 of 100 μm to 150 μm e.g., 100 μm to 140 μm, 100 μm to 130 μm, 100 μm to 125 μm,
a Dv10 of 20 μm to 40 μm e.g., 20 μm to 30 μm, 20 μm to 25 μm,
an average specific surface area of 0.05 to 0.3 m²/g e.g., 0.1 to 0.28 m²/g, 0.1 to 0.25 m²/g, 0.20 to 0.21 m²/g, e.g., 0.15 m²/g, 0.2 m²/g, 0.21 m²/g, 0.206 m²/g, 0.25 m²/g, wherein the specific surface area is measured by gas e.g., nitrogen or krypton gas adsorption e.g., as determined using the N2-BET or Kr-BET adsorption method, for instance as described in Example 3, and
a drug loading of 11.5 to 13%, e.g., 11.5 w/w % to 12.5 w/w % e.g., 12 w/w % to 12.5 w/w %.

In another embodiment the biodegradable polymer microparticles comprised in the composition of the invention comprise a biodegradable polymer and a somatostatin analogue, preferably octreotide, or a pharmaceutically acceptable salt thereof, wherein said biodegradable polymer consists of PLGA, the PLGA having:
a molar ratio of lactide to glycolide of 83:17 to 87:13,
an inherent viscosity of 0.25 to 0.35 dl/g as measured at a concentration of 0.5 w/w % in chloroform at 25° C.; and
said biodegradable polymer microparticles have:
a Dv50 of 60 μm to 75 μm,
an average specific surface area of 0.05 to 0.3 m²/g, wherein the specific surface area is measured by gas e.g., nitrogen or krypton gas adsorption e.g., as determined using the N2-BET or Kr-BET adsorption method, for instance as described in Example 3, and
a drug loading of 11.5 to 13%.

In another embodiment the biodegradable polymer microparticles comprised in the composition of the invention comprise a biodegradable polymer and a somatostatin analogue, preferably octreotide, or a pharmaceutically acceptable salt thereof, wherein said biodegradable polymer consists of PLGA, the PLGA having:
a molar ratio of lactide to glycolide of 83:17 to 87:13,
an inherent viscosity of 0.25 to 0.35 dl/g as measured at a concentration of 0.5 w/w % in chloroform at 25° C.; and
said biodegradable polymer microparticles have:
a Dv50 of 60 μm to 70 μm,
an average specific surface area of 0.1 to 0.28 m²/g, wherein the specific surface area is measured by gas e.g., nitrogen or krypton gas adsorption e.g., as determined using the N2-BET or Kr-BET adsorption method, for instance as described in Example 3, and
a drug loading of 11.5 w/w % to 12.5 w/w %.

In another embodiment the biodegradable polymer microparticles comprised in the composition of the invention comprise a biodegradable polymer and a somatostatin analogue, preferably octreotide, or a pharmaceutically acceptable salt thereof, wherein said biodegradable polymer consists of PLGA, the PLGA having:
a molar ratio of lactide to glycolide of 83:17 to 87:13,
an inherent viscosity of 0.25 to 0.35 dl/g as measured at a concentration of 0.5 w/w % in chloroform at 25° C.; and
said biodegradable polymer microparticles have:
a Dv50 of 62 μm to 65 μm,
an average specific surface area of 0.1 to 0.25 m²/g e.g., 0.15 m²/g, 0.2 m²/g, 0.20 m²/g, 0.206 m²/g, 0.21 m²/g, 0.25 m²/g, wherein the specific surface area is measured by gas e.g., nitrogen or krypton gas adsorption e.g., as determined using the N2-BET or Kr-BET adsorption method, for instance as described in Example 3, and
a drug loading of 12 w/w % to 12.5 w/w %.

In another embodiment the biodegradable polymer microparticles comprised in the composition of the invention comprise a biodegradable polymer and a somatostatin analogue, preferably octreotide, or a pharmaceutically acceptable salt thereof, wherein said biodegradable polymer consists of PLGA, the PLGA having:
a molar ratio of lactide to glycolide of 83:17 to 87:13,
an inherent viscosity of 0.25 to 0.35 dl/g as measured at a concentration of 0.5 w/w % in chloroform at 25° C.; and
said biodegradable polymer microparticles have:
a Dv50 of 60 μm to 75 μm,
a Dv90 of 100 μm to 150 μm,
a Dv10 of 20 μm to 40 μm,
an average specific surface area of 0.05 to 0.3 m²/g, wherein the specific surface area is measured by gas e.g., nitrogen or krypton gas adsorption e.g., as determined using the N2-BET or Kr-BET adsorption method, for instance as described in Example 3, and
a drug loading of 11.5 to 13%.

In another embodiment the biodegradable polymer microparticles comprised in the composition of the invention comprise a biodegradable polymer and a somatostatin analogue, preferably octreotide, or a pharmaceutically acceptable salt thereof, wherein said biodegradable polymer consists of PLGA, the PLGA having:
a molar ratio of lactide to glycolide of 83:17 to 87:13,
an inherent viscosity of 0.25 to 0.35 dl/g as measured at a concentration of 0.5 w/w % in chloroform at 25° C.; and
said biodegradable polymer microparticles have:
a Dv50 of 60 μm to 70 μm,
a Dv90 of 100 μm to 140 μm,
a Dv10 of 20 μm to 30 μm,
an average specific surface area of 0.1 to 0.28 m²/g, wherein the specific surface area is measured by gas e.g., nitrogen or krypton gas adsorption e.g., as determined using the N2-BET or Kr-BET adsorption method, for instance as described in Example 3, and
a drug loading of 11.5 w/w % to 12.5 w/w %.

In another embodiment the biodegradable polymer microparticles comprised in the composition of the invention comprise a biodegradable polymer and a somatostatin analogue, preferably octreotide, or a pharmaceutically acceptable salt thereof, wherein said biodegradable polymer consists of PLGA, the PLGA having:
a molar ratio of lactide to glycolide of 83:17 to 87:13,
an inherent viscosity of 0.25 to 0.35 dl/g as measured at a concentration of 0.5 w/w % in chloroform at 25° C.; and
said biodegradable polymer microparticles have:
a Dv50 of 62 μm to 65 μm,
a Dv90 of 100 μm to 130 μm, e.g., 100 μm to 125 μm,
a Dv10 of 20 μm to 25 μm,
an average specific surface area of 0.1 to 0.25 m²/g e.g., 0.15 m²/g, 0.2 m²/g, 0.20 m²/g, 0.206 m²/g, 0.21 m²/g, 0.25 m²/g, wherein the specific surface area is measured by gas e.g., nitrogen or krypton gas adsorption e.g., as determined using the N2-BET or Kr-BET adsorption method, for instance as described in Example 3, and
a drug loading of 12 w/w % to 12.5 w/w %.

A somatostatin analogue, or pharmaceutically acceptable salt thereof, is comprised in the biodegradable polymer microparticles e.g., biodegradable polymer microspheres, comprised in the pharmaceutical composition of the invention. Said somatostatin analogue, or pharmaceutically acceptable salt thereof, may be dispersed e.g., homogenously dispersed, within the internal continuous biodegradable polymer e.g., PLGA, matrix of the biodegradable polymer microparticles e.g., biodegradable polymer microspheres.

The term “somatostatin analogue” as used herein refers to any pharmaceutically active synthetic (as opposed to naturally occurring) compound designed to pharmacologically mimic somatostatin with respect to binding to one or more of the five somatostatin receptor subtypes. A somatostatin analogue may be considered pharmaceutically active if it binds to at least one somatostatin receptor subtype, for example if it binds to at least one of the five somatostatin receptor subtypes in the nM or μM range e.g., if the IC50 (nM) is less than 70 e.g., less than 60, less than 50, less 40, less than 30, less than 20, less than 10 e.g., less than 6 e.g., less than 5, less than 4, less than 3, less than 2, less than 1, for one or more of the five somatostatin subtypes. Preferably the IC50 (nM) is less than 6 for one or more of the five somatostatin subtypes e.g., less than 6 for subtype 2.

The IC50 ((nM) may be determined by known methods e.g., by in vivo measuring the binding of an ¹²⁵I labelled somatostatin analogue to the cloned subtype receptor of interest e.g., by measuring the binding in CHO-K1 (Chinese hamster ovary) cells transfected with the receptor of interest e.g., subtype 2.

The term “somatostatin” as used herein refers to any naturally occurring somatostatin e.g., somatostatin-14 or somatostatin-28.

Non limiting examples of somatostatin analogues include octreotide, lanreotide, pasireotide, and vapreotide.

Any pharmaceutically acceptable salt of a somatostatin analogue may be comprised in the biodegradable polymer microparticles e.g., biodegradable polymer microspheres, comprised in the pharmaceutical composition of the invention. Acceptable salts may be an acid addition salt with e.g., inorganic acid, polymeric acid or organic acid, for example with hydrochloric acid, acetic acid, lactic acid, citric acid, fumaric acid, malonic acid, maleic acid, tartaric acid, aspartic acid, benzoic acid, succinic acid or pamoic (embonic) acid. Said acid addition salts may be mono or divalent salts. Said salts may be water soluble or non-water soluble.

In a specific embodiment the somatostatin analogue comprised in the biodegradable polymer PLGA microparticles e.g., biodegradable polymer microspheres, comprised in the pharmaceutical composition of the invention is octreotide or a pharmaceutically acceptable salt thereof.

Preferred pharmaceutically acceptable salts may be the acetate or pamoate salts.

In a more specific embodiment, the somatostatin analogue is octreotide and/or a pharmaceutically acceptable salt thereof, wherein said pharmaceutically acceptable salt thereof is selected from the group consisting of octreotide acetate, octreotide pamoate, and a combination thereof. Preferably said pharmaceutically acceptable salt thereof is octreotide acetate.

An advantage of the sustained release pharmaceutical composition of the invention is that only biodegradable polymer microparticle defined herein and formed from PLGA defined herein are needed in the composition to achieve a composition having one or more of:
a desired sustained release e.g., 30 days or more e.g., 60 days or more, 90 days or more, or 120 days or more,
a low initial release (burst), and
a surprisingly high bioavailability, of the somatostatin analogue, or pharmaceutically acceptable salt thereof, following administration to a patient.

Accordingly, the biodegradable polymer used to form the biodegradable polymer microparticles comprised in the pharmaceutical composition of the invention may comprise, or may substantially comprise, only the PLGA defined herein.

In an embodiment the biodegradable polymer comprises at least 50% of PLGA defined herein e.g., PLGA having a molar ratio of lactide to glycolide of 80:20 to 90:10, and an inherent viscosity of 0.2 to 0.4 dl/g as measured at a concentration of 0.5 w/w % in chloroform at 25° C.

In a more specific embodiment at least 85 w/w % e.g., least 90 w/w %, at least 95 w/w % or 100 w/w % of said biodegradable polymer may be PLGA as defined herein. Preferably 100% of the biodegradable polymer is PLGA as defined herein.

The wording “at least 50%” as used hereinabove and below and especially in the preceding two paragraphs as well as the following paragraph may refer in specific embodiments to at least 60 w/w %, at least 65 w/w %, at least 70 w/w %, at least 75 w/w %, at least 80 w/w %, at least 85 w/w %, at least 90 w/w % e.g., at least 95 w/w %, such as 96 w/w % or more, 97 w/w % or more, 98 w/w % or more, 99 w/w % or more, e.g., 100 w/w %.

In the above embodiments with the biodegradable polymer comprising less than 100% PLGA as defined herein, the remainder to 100% is preferably any one or more of the biodegradable polymers that are listed in the above paragraph defining the term “biodegradable polymer” (with the exception of the PLGA as defined herein).

The biodegradable polymer microparticles described herein may be the only biodegradable polymer microparticles comprised in the composition of the invention, alternatively, they may make up at least 50 w/w % of the total of any biodegradable polymer microparticles comprised in the composition e.g., at least 60 w/w %, at least 70 w/w %, at least 80 w/w %, at least 90 w/w %, at least 95 w/w %, at least 99 w/w %, at least 99.9 w/w %. Preferably the biodegradable polymer microparticles described herein make up at least 80 w/w %, at least 85 w/w % e.g., at least 90 w/w %, at least 95 w/w %, at least 99 w/w %, at least 99.9 w/w %. Most preferably the biodegradable polymer microparticles described herein are the only biodegradable polymer microparticles comprised in the composition of the invention.

If the biodegradable polymer microparticles make up less than 100 w/w % of the biodegradable polymer microparticles comprised in the pharmaceutical composition of the invention, the remaining % of biodegradable microparticles are “other” biodegradable microparticles to those defined herein. Said other biodegradable microparticles may also comprise PLGA. Said PLGA may be the same or different to the PLGA defined herein. Said other biodegradable microparticles may also comprise a somatostatin analogue, or pharmaceutically acceptable salt thereof. Preferably said other biodegradable polymer microparticles are formed from biodegradable polymer comprising PLGA defined herein, and preferably they comprise a somatostatin analogue, or pharmaceutically acceptable salt thereof, as defined herein. More preferably said other biodegradable polymer microparticles are formed from biodegradable polymer comprising at least 50 w/w PLGA defined herein, and preferably they comprise a somatostatin analogue, or pharmaceutically acceptable salt thereof, as defined herein. The other biodegradable polymer microparticles may differ from the biodegradable polymer microparticles defined herein because they have a different drug loading, DV50 or specific surface area. Said other biodegradable polymer microparticles may only differ by one of these attributes/characteristics.

The composition according to the present invention may additionally comprise one or more other pharmaceutical excipients e.g., pharmaceutical excipients ordinarily comprised in the type of formulation in question e.g., diluents, surfactants, stabilisers, release modifiers, preservatives, antioxidants, buffers, anti-agglomerating agents and the like.

Non limiting examples of other pharmaceutical excipients include polyvinylalcohol, polyvinyl pyrrolidone, carboxymethyl cellulose sodium (CMC-Na), dextrin, polyethylene glycol, suitable surfactants such as poloxamers, also known as poly(oxyethylene-block-oxypropylene), Poly(oxyethylene)-sorbitan-fatty acid esters known and commercially available under the trade name TWEEN® (e.g., Tween 20, Tween 40, Tween 60, Tween 80, Tween 65 Tween 85, Tween 21, Tween 61, Tween 81), Sorbitan fatty acid esters e.g. of the type known and commercially available under the trade name SPAN, Lecithins, inorganic salts such as zinc carbonate, magnesium hydroxide, magnesium carbonate, or protamine, e.g., human protamine or salmon protamine, or natural or synthetic polymers bearing amine-residues such as polylysine, hydroxyethyl cellulose (HEC) and/or hydroxypropyl cellulose (HPC), Polyvinyl pyrolidone), and gelatine e.g., porcine or fish gelatine. Suitable anti-agglomerating agents include, for example, mannitol, glucose, dextrose, sucrose, sodium chloride, or water-soluble polymers such as polyvinyl alcohol, polyvinyl pyrrolidone or polyethylene glycol.

The pharmaceutical composition of the invention can provide a sustained release of the somatostatin analogue, or pharmaceutically acceptable salt thereof, comprised within the biodegradable polymer microparticles comprised therein.

The term “sustained release” as used herein refers to the continuous release of an amount of a somatostatin analogue, or pharmaceutically acceptable salt thereof, over, or substantially over, a predetermined release period (e.g., 30 days or more, 60 days or more, 90 days or more, 120 days or more), as measured in-vivo e.g., after administration by IM injection in a patient. The amount of the somatostatin analogue release may be an effective amount. For example, in one embodiment, sustained release may be confirmed by administering a dose of 30 mg somatostatin analogue, preferably octreotide, or pharmaceutically acceptable salt thereof, to a patient by IM injection and confirming that the plasma level of the somatostatin analogue and especially octreotide does not fall to a level below 0.5 ng/mL for a period of at least 30 days.

In another embodiment wherein the somatostatin analogue is octreotide or a pharmaceutically acceptable salt thereof, sustained release may be confirmed by administering by IM injection to a patient e.g., a patient in need thereof e.g., a patient suffering from acromegaly or GEP-NETs, an amount of the composition of the invention corresponding to a dosage of octreotide of 90 mg, and confirming that the plasma level of octreotide does not fall to a level below 0.5 ng/mL over the period from day 21 to day 84 following administration on day zero.

In this test and also in other tests described hereinabove and below, it is possible to conduct the test with a larger number of patients, e.g., 5, 10, 15, 20, 50 or 100 patients followed by averaging the results, to thereby improve quality and precision of the obtained result.

An effective amount in the context of the invention may be an amount that results in a patient having a therapeutically effective plasma level of a somatostatin analogue e.g., octreotide, and may depend on a patient's existing plasma level.

An effective amount may be an amount that results in a somatostatin analogue plasma level of 0.1 ng/mL e.g., 0.2, 0.3, 0.4, 0.5, 0.6, 0.7, 0.8, 0.9, 1 ng/mL or higher.

In the case of a somatostatin analogue e.g., lanreotide, octreotide, pasireotide, or vapreotide, a therapeutically effective plasma level may be, for example be 0.3 ng/mL to 12 ng/mL or higher e.g., 2 ng/mL, 2.5 ng/mL, 3 ng/mL, 3.5 ng/mL, 4 ng/mL, 4.5 ng/mL, 5 ng/mL, 5.5 ng/mL, 6 ng/mL, 6.5 ng/mL, 7 ng/mL, 7.5 ng/mL, 8 ng/mL, 8.5 ng/mL, 9 ng/mL, 9.5 ng/mL 10 ng/mL or higher.

In the case of octreotide a therapeutically effective plasma level may be, for example, 0.3 ng/mL to 1 ng/mL or higher e.g., 0.3 ng/mL to 3 ng/mL e.g., 0.4 ng/mL, 0.5 ng/mL, 0.6 ng/mL, 0.7 ng/mL, 0.8 ng/mL, 0.9 ng/mL, 1.5 ng/mL, 1.8 ng/mL, 2 ng/mL, 2.5 ng/mL, 3 ng/mL. A particularly effective plasma level may be 1 ng/mL.

What constitutes a therapeutically effective plasma level of a somatostatin analogue may depend on the characteristics of the patient e.g., how well the patient responds to the somatostatin analogue in question.

The term “therapeutic effect or therapeutically effective” as used herein refers not only to complete cure of a disease, but also to the alleviation or amelioration of a disease or any symptom related thereto e.g., improvement in quality of life, or a slowing of disease progression or stabilisation of the disease. The therapeutic effect in a patient may be monitored by any method known to those with ordinary skill in the clinical art of treating the disease in question.

In some embodiments of the invention, an effective amount of a composition of the invention, may be an amount that results in a somatostatin analogue, e.g., octreotide, plasma level that leads to a reduction in insulin-like growth factor 1 (IGF1) serum levels in a patient e.g., a patient suffering from acromegaly. Administration of an effective amount e.g., of a composition of the invention comprising octreotide or a pharmaceutically acceptable salt thereof, may result in a reduction in serum IGF1 levels. There is no particular limit to the reduction that can be accomplished. In some embodiments, said reduction may be up to 50% from baseline (the IGF1 level in a patient before administration of the pharmaceutical composition of the invention) e.g., up to 40%, or up to 30%, while the reduction is at least 1%, typically at least 15% and in particular at least 20%. For these determinations, serum IGF1 levels may be measured using a validated IDS-iSYS method or a validated LC-MS/MS method such as the method set out in Example 10B herein.

In some embodiments of the invention, an effective amount of a composition of the invention, may be an amount that results in a somatostatin analogue, e.g., octreotide, plasma level that leads to a reduction in growth hormone (GH) serum levels in a patient e.g., a patient suffering from acromegaly. Administration of an effective amount e.g., of a composition of the invention comprising octreotide or a pharmaceutically acceptable salt thereof, may in some embodiments result in the decrease of GH serum levels e.g., to a value of 3 μg/L or less, e.g., 2.5 μg/L or less, e.g., 2 μg/L or less, e.g., 1.5 μg/L or less, or e.g., 1 μg/L or less, the minimum decrease being the level of detection. For these determinations, serum Human Growth Hormone (GH) levels may be determined using the IDS-iSYS Multi-Discipline Automated System described in more detail hereinbelow.

In a preferred embodiment an effective amount of a composition of the invention, may be an amount that results in a somatostatin analogue e.g., octreotide, plasma level that leads to a reduction in IGF1 and/or GH serum levels in a patient suffering from acromegaly. Said reductions may be as described above.

In some embodiments of the invention, an effective amount of a composition of the invention, may be an amount that results in a somatostatin analogue, e.g., octreotide, plasma level that leads to a reduction in chromogranin A (CgA) serum levels in a patient e.g., a patient suffering from GEP-NETs. There is no particular limit to the reduction that can be accomplished. CgA serum levels may be determined using the Time Resolved Amplified Cryptate Emission (TRACE) assay of the manufacturer BRAHMS, using the B⋅R⋅A⋅H⋅M⋅S CgA II KRYPTOR assay kit (Ref. No. 839.050).

A sustained release e.g., of an effective amount of a somatostatin analogue, substantially over a predetermined release period may refer to release over a predetermined release period minus a lag period e.g., a period following injection before the release of effective amounts is reached e.g., a lag period may be 21 days or less, 18 days or less, 15 days or less, e.g., 14 days or less, 10 days or less, or 5 days or less. For example, if the predetermined release period is 90 days and the lag period is 5 days, after administration of a first dose of the composition of the present invention at day 0, the patient's plasma level of somatostatin analogue will be within the therapeutically effective range from day 5 until the end of the sustained release period. Typically, a lag period will only be seen in a patient that does not have therapeutically effective plasma levels of the somatostatin analogue and is being administered a composition of the invention for the 1^{st} time, e.g., a patient in need of treatment who has not been pre-treated with a somatostatin analogue.

The term “patient” as used herein refers to a human or animal subject. In an embodiment of the invention the patient is a human. The human may be an adult or child. Prior to an administration of the composition of the invention, the patient may have been pre-treated (previously treated) with a somatostatin analogue, or pharmaceutically acceptable salt thereof, and may have therapeutically effective plasma levels of said somatostatin analogue e.g., of octreotide or lanreotide (the patient may for example, be switching from one type of somatostatin analogue treatment to treatment with the composition of the invention, or may have been pre-treated to ensure the somatostatin analogue is well supported by the patient). Therapeutically effective plasma levels of a somatostatin analogue may be present for several weeks after the last administration of any pre-treatment. The patient may also be pre-treated so as to avoid any lag period following the 1^{st} administration of a composition of the invention.

The pharmaceutical composition of the invention preferably provides a sustained release of the somatostatin analogue, or a pharmaceutically acceptable salt thereof, over 30 days or more e.g., over 40 days or more, over 50 days or more, over 60 days or more, over 70 days or more, over 80 days or more, over 90 days or more, over 100 days or more, over 110 days or more, over 120 days or more, over 150 days or more, over 180 days or more e.g., over 200 days.

30 days or more may refer to 30 to 200 days e.g., 30 to 180 days e.g., 30 days to 126 days e.g., 30 days to 120 days e.g., 30 days to 90 days, e.g., 30 days to 60 days e.g., 56 days, 30 to 50 days e.g., 49 days, 42 days, 30 to 40 days e.g., 35 days, 30 days.

60 days or more may refer to a 60 to 200 days e.g., 60 to 180 days e.g., 60 to 126 days e.g., 60 to 120 days e.g., 60 to 90 days e.g., 60 to 80 days, 77 days, 84 days, 60 to 70 days e.g., 60 days, 63 days, 70 days.

90 days or more may refer to 90 to 200 days e.g., 90 to 180 days e.g., 90 to 126 days e.g., 90 to 120 days e.g., 112 days, 90 to 110 days e.g., 105 days, 90 to 100 days e.g., 90 days, 91 days, 98 days.

120 days or more may refer to 120 to 200 days e.g., 120 to 180 days e.g., 120 to 126 days e.g., e.g., 120 days.

The sustained release periods disclosed herein are given in days. However, such periods may also be equated to the same periods in weeks or months e.g., 28-30 days may equate to 4 to 5 weeks or to 1 month, 56-60 days may equate to 8 to 9 weeks or 2 months, 84-90 days may equate to 12 to 13 weeks or 3 months, and 112-120 days may equate to 17 to 18 weeks or 4 months, 140-150 days may equate to 21 to 22 weeks or 5 months, 168-180 days may equate to 25 to 26 weeks or 6 months, and 196-200 days may equate to 27 to 28 weeks or 7 months etc.

In an embodiment the pharmaceutical composition of the invention provides a sustained release of the somatostatin analogue, or a pharmaceutically acceptable salt thereof, over 30 days or more, preferably over 30 to 200 days e.g., 30 to 180 days e.g., 30 to 126 days, or, in other embodiments, over about 30 days or more, preferably over about 30 to 200 days e.g., 28 to 200 days e.g., 30 to 180 days e.g., 30 to 126 days. In a more specific embodiment, the pharmaceutical composition of the invention is a 1 month sustained release formulation, preferably administered to a patient once about every 30 days e.g., every 24-35 days e.g., 24 days, 25 days, 26 days, 27 days, 28 days, 29 days, 30 days, 31 days, 32 days, 33 days, 34 days.

In an embodiment of the invention the sustained release pharmaceutical composition of the invention provides a sustained release over 60 days or more, preferably over 60 to 200 days e.g., 60 to 180 days e.g., 60 days to 126 days, or, in other embodiments, over about 60 days or more, preferably over 56 to 200 e.g., 60 to 200 days e.g., 60 to 180 days e.g., 60 days to 126 days. In a more specific embodiment, the pharmaceutical composition of the invention is a 2 month sustained release formulation, preferably administered to a patient one about every 60 days e.g. every 54-65 days e.g., 54 days, 55 days, 56 days, 57 days, 58 days, 59 days, 60 days, 61 days, 62 days, 63 days, 64 days.

In an embodiment, the sustained release pharmaceutical composition of the invention provides a sustained release over 90 days or more, preferably over 90 to 200 days e.g., 90 to 180 days e.g., 90 days to 126 days, or, in other embodiments, over about 90 days or more, preferably over 84 to 200 days e.g., 90 to 200 days e.g., 90 to 180 days e.g., 90 days to 126 days. In a more specific embodiment, the pharmaceutical composition of the invention is a 3 month sustained release formulation, preferably administered to a patient once about every 90 days e.g. every 84-95 days e.g., 84 days, 85 days, 86 days, 87 days, 88, days, 89 days, 90 days, 91 days, 92 days, 93 days, 94 days.

In an embodiment the pharmaceutical composition of the invention provides a sustained release of the somatostatin analogue, or a pharmaceutically acceptable salt thereof, over 120 days or more, preferably over 120 to 200 days e.g., 120 to 180 days e.g., 120 to 126 days, or, in other embodiments, over about 120 days or more, preferably over 112 to 200 days e.g., 120 to 200 days e.g., 120 to 180 days e.g., 120 to 126 days. In a more specific embodiment, the pharmaceutical composition of the invention is a 4 month sustained release formulation, preferably administered to a patient once about every 120 days e.g., every 112-125 days e.g., 114-125 days e.g., 112 days, 113 days, 114 days, 115 days, 116 days, 117 days, 118 days, 119 days, 120 days, 121 days, 122 days, 123 days, 124 days.

Whilst the pharmaceutical composition described herein may provide a sustained release as described herein above and below, said pharmaceutical composition is not limited to being a sustained release composition as described herein. The product features of the pharmaceutical composition as specified herein e.g., in appended claim 1 give rise to advantageous release characteristics irrespective whether these are categorized as “sustained release” of any specific type or not.

The pharmaceutical composition of the invention may exhibit a low initial release (low burst) of the somatostatin analogue, or pharmaceutically acceptable salt thereof, following administration to a patient. This may serve to minimise any side effects/adverse reactions that may be associated with administration of said somatostatin analogue, or pharmaceutically acceptable salt thereof. A pharmaceutical composition of the invention may be considered as having a low burst if it is characterized by a release of the somatostatin analogue (comprised therein) that fulfils the following criteria: release of less than 15% e.g., less than 10%, of the somatostatin analogue e.g., octreotide, comprised therein over 5 hours. Wherein said release is measured in vitro (at 37° C. in 900 mL of a pH4 100 mM acetate buffer e.g., stirring at 75 RPM) according to the method described in the European Pharmacopoeia 10, 2.9.3. and wherein said composition is tested in an amount equating to 30 mg, 60 mg or 90 mg of the somatostatin analogue e.g., octreotide. The percentage indications in this paragraph and the following two paragraphs may be understood as expressed on a w/w-basis or, alternatively, on a mol/mol-basis.

In an embodiment, the pharmaceutical composition of the present invention releases less than 7% e.g., less than 6%, 5%, 4%, 3%, of the somatostatin analogue e.g., octreotide, comprised therein over 5 hours.

In a more specific embodiment of the invention the pharmaceutical composition of the invention releases over 5 hours less than 3% of the somatostatin analogue e.g., octreotide, comprised therein, as measured in vitro at 37° C. in an acetate buffer at pH4.

The pharmaceutical composition according to the present invention may be a composition of dried biodegradable polymer microparticles e.g., biodegradable polymer microspheres, ready for suspension in a liquid vehicle prior to injection, or it may e.g., be a ready to use liquid vehicle suspension comprising biodegradable polymer e.g., biodegradable polymer microspheres. Said liquid vehicle may be an aqueous (water-based) or non-aqueous vehicle. The biodegradable polymer microparticles e.g., biodegradable polymer microspheres contained in said liquid vehicle may be any of the biodegradable polymer microparticles, e.g., biodegradable polymer microspheres, as described hereinabove.

In an embodiment, the pharmaceutical composition of the invention is a liquid vehicle suspension comprising biodegradable polymer microparticles e.g., biodegradable polymer microspheres, or is a composition of dried biodegradable polymer microparticles e.g., biodegradable polymer microspheres. Said liquid vehicle may be an aqueous (water-based) or non-aqueous liquid vehicle.

The composition of dried biodegradable polymer microparticles e.g., dried biodegradable polymer microspheres, or liquid vehicle suspension comprising the biodegradable polymer microparticles e.g., biodegradable polymer microspheres, may be provided in a syringe e.g., ready for injection.

The composition of dried biodegradable polymer microparticles or said liquid vehicle suspension comprising biodegradable polymer microparticles may contain the biodegradable polymer microparticles defined herein as the only biodegradable polymer microparticles of the composition or may be a mix of the biodegradable polymer microparticles defined herein and other biodegradable polymer microparticles. As previously indicated, preferably at least 50 w/w %, more preferably at least 80 w/w %, of the biodegradable polymer microparticles comprised in such a composition of the invention are biodegradable polymer microparticles defined herein.

The term aqueous liquid vehicle or water-based liquid vehicle as used herein refers to a pharmaceutically acceptable liquid that is predominately water e.g., a pharmaceutically acceptable liquid that comprises more than 50% water e.g., 60% or more, 70% or more, 80% or more, 90% or more, 93% or more, 95% or more, or even 97% or more of water. These percentage indications are (w/v) percentages.

A water-based (aqueous) liquid vehicle may contain pharmaceutical excipients. Said excipients may be used to ensure isotonicity and to improve the wettability and non-sedimentation properties of the microparticles e.g., microspheres. Examples of such pharmaceutical excipients include, but are not limited to, mannitol, sodium chloride, glucose, dextrose, sucrose, or glycerines, non-ionic surfactants e.g., poloxamers, poly(oxyethylene)-sorbitan-fatty acid esters, carboxymethyl cellulose sodium (CMC-Na), sorbitol, poly(vinylpyrrolidone), or aluminium monostearate, polyethylene glycol (PEG) e.g., PEG 4000.

The composition of dried biodegradable polymer microparticles e.g., dried biodegradable polymer microspheres, can be suspended e.g., prior to injection, in a water-based vehicle e.g., in a water-based vehicle comprising glucose and PEG 4000 e.g., 3 to 7 w/w % glucose e.g., 5 w/w % glucose, and 5 to 9 w/w % e.g., 7.5 w/w % PEG 4000, or, for example in a water-based vehicle comprising sodium carboxymethyl cellulose, mannitol, and tween 80 e.g., 1 w/w % to 2 w/w % carboxymethyl cellulose, 4 to 5 w/w % mannitol, and 0.05 to 0.15 w/w % tween 80.

Advantageously, the inventors have found that the somatostatin analogue release profile of a non-aqueous liquid vehicle suspension comprising biodegradable polymer microparticles in accordance with the invention may be the same, or substantially the same, as that of a corresponding (all characteristics of the microparticles being the same) water-based vehicle suspension e.g., there is no change, or substantial change, in the burst, lag, or release period, meaning that non-aqueous and aqueous liquid vehicles can be interchangeably employed depending on the need e.g., because of regulatory constraints, financial considerations or personal preference.

The term “non-aqueous liquid” as used herein refers to a pharmaceutically acceptable liquid that is free from or substantially free from water e.g., a pharmaceutically acceptable liquid that comprises 0.25% (w/v) water or less, 0.1% water or less, 0.05% (w/v) water or less. The non-aqueous liquid is inert or substantially inert with respect to the biodegradable polymer microparticles comprised in the composition of the invention, as well as with respect to any somatostatin analogue comprised therein e.g., it does not interact, or substantially interact, with, or solubilise, or substantially solubilise, the biodegradable polymer e.g., PLGA, microparticles or somatostatin analogue comprised in them.

The water content of a non-aqueous liquid may be measured using conventional methods e.g., The Micro Determination Method as described in “European Pharmacopoeia”, 10.0, chapter 2.5.32.

In an embodiment of the invention the non-aqueous liquid has a viscosity of 25 to 33 mPa at 20° C.

The viscosity of a non-aqueous liquid may be measured using conventional methods e.g., The Capillary Viscometer Method as described in “European Pharmacopoeia”, 10.0, chapter 2.2.9.

The non-aqueous liquid may be a pharmaceutically acceptable oil. The term oil as used herein refers to a substance that is in a viscous liquid state at ambient temperatures (20° C.-30° C.), or slightly warmer, and is both hydrophobic (immiscible with water) and lipophilic (miscible with other oils). Non limiting examples of suitable oils include vegetable oils e.g., coconut oil, palm oil, palm kernel oil, sesame oil, soybean oil, almond oil, rapeseed oil, corn oil, sunflower oil, peanut oil, olive oil, castor oil, soybean oil, safflower oil, cottonseed oil, ethyl oleate, and any combinations thereof.

In an embodiment of the invention the non-aqueous liquid is a pharmaceutically acceptable oil. Particularly suitable oils may comprise medium chain triglycerides (MCTs).

The term medium chain triyglceride (MCT) as used herein refers to glyceride esters formed from glycerol and three medium chain fatty acids; each of the three medium chain fatty acids being a C6 to C12 fatty acid i.e. a carboxylic acid with an aliphatic chain of 6 to 12 carbon atoms e.g., C6 (hexanoic acid), C8 (octanoic acid), C10 (decanoic acid), C12 (dodecanoic acid). The three medium chain fatty acids from which the triglyceride is formed may all be the same e.g., all three may be medium chain fatty acids with aliphatic chains of, for example, 8 or 10 carbons atoms, or one or all of said medium chain fatty acids may be different from the others. The aliphatic chains of the MCTs may be saturated or unsaturated.

In an embodiment the non-aqueous liquid is a pharmaceutically acceptable oil essentially consisting of, one or more medium chain triglycerides (MCTs).

A pharmaceutically acceptable oil is considered to essentially consist of one or more MCT if it comprises more than 50% of one or more medium chain triglyceride e.g., 60% or more, 65% or more, 70% or more, 75% or more, 80% or more, 85% or more, 90% or more, 95% or more, e.g, 96%, 97%, 98%, 99%, 100. Wherein the content of a specific MCT in the oil is measured by GC in accordance with the method set out in Ph. Eur. 2.4.22, without further conversion.

In an even more specific embodiment the non-aqueous liquid is an oil essentially consisting of C8 and/or C10 medium chain triglycerides e.g., essentially consisting of C8 and C10 medium chain triglycerides e.g. consisting of 50% to 80% of C8 MCTs, and 20% to 50% C10 MCTs such as 58% C8 MCTs and 41% C10 MCTs as it is present in MIGLYOL® 812 as mentioned below. As stated above, the MCT content of the oil is measured by GC in accordance with the method set out in Ph. Eur. 2.4.22, without further conversion.

For stability reasons, it is preferred if MCTs employed in the composition of the invention are saturated e.g., at least 80% of any MCTs employed in the composition are saturated e.g., at least 85%, at least 90%, at least 95%, at least 99%, at least 99.5% e.g., 100%. Wherein the MCT content of the oil is measured by GC in accordance with the method set out in Ph. Eur. 2.4.22, without further conversion.

Pharmaceutically acceptable MCT oils are commercially available. Non limiting examples of commercially available MCT oils include MIGLYOL® 810, 812, 818, (Sasol Germany GmbH, Witten, Germany).

A particularly effective MCT oil may be MIGLYOL® 812.

The amount of a liquid vehicle used for suspension (v) may be about 1 to about 5 ml per dose, e.g., about 2 to about 3 ml per dose e.g., 2.9 ml, 2.8 ml, 2.7 ml, 2.6 ml, e.g., about 2 to about 2.5 ml per dose e.g., 2.5 ml, 2.4 ml. In one embodiment, the composition of dried biodegradable polymer microparticles e.g., biodegradable polymer microspheres, and a vehicle for suspension may be housed separately in a double chamber syringe.

The liquid vehicle used for suspension may comprise biodegradable polymer microparticles in any suitable concentration depending on the desired dose and the desired injection volume. In an embodiment the liquid vehicle comprises biodegradable polymer microparticles in a concentration of 100 mg/mL to 500 mg/mL e.g., 100 to 400 mg/mL e.g., 200 to 400 mg/mL e.g., 230 to 350 mg/mL, 200 to 350 mg/mL e.g., 250 to 350 mg/mL based on the total volume of the suspension. Preferably the liquid vehicle comprises biodegradable polymer microparticles in a concentration of 100 mg/mL to 375 mg/mL e.g., 125 mg/mL to 375 mg/ml, 250 to 375 mg/mL, based on the total volume of the suspension. The injection volume of the liquid suspension may be about 1 to about 5 ml per dose, e.g., about 2 to about 2.5 ml per dose e.g., 2.4 mL per dose.

A liquid suspension of a composition of dried biodegradable polymer microparticles in accordance with the invention, or a ready to use liquid suspension comprising biodegradable polymer microparticles in accordance with the invention, may be administered via a needle of an appropriate gauge (G) e.g., 16G or smaller e.g., 18G, 19G, 20G, 21G, 23G, 25G, preferably 20G.

A pharmaceutical composition in accordance with the present invention can be manufactured aseptically, or non-aseptically and then sterilised terminally e.g., by irradiation e.g., by X-ray or gamma irradiation. In an embodiment the pharmaceutical composition of the invention is sterilised by irradiation.

A pharmaceutical composition of the invention may be manufactured by any known method for making the composition in question.

Biodegradable polymer microparticles e.g., biodegradable polymer microspheres, comprising a somatostatin analogue, or pharmaceutically acceptable salt thereof, may be manufactured by any processes known in the art, e.g., coacervation or phase separation, spray drying, water-in-oil (W/O) or water-in-oil-in-water (W/O/W) or solids-in-oil-in-water (S/O/W), or oil-in-water (O/W) emulsion/suspension methods followed by solvent extraction or solvent evaporation.

In an embodiment of the invention the biodegradable polymer microparticles are formed from biodegradable polymer comprising at least 50 w/w % e.g., at least 85 w/w % e.g., 100% PLGA as defined herein, and the biodegradable polymer microparticles e.g., microspheres comprising a somatostatin analogue, or pharmaceutically acceptable salt thereof, are manufactured by/are obtainable by, a method/process comprising the following steps:
i. Preparing an organic phase comprising an organic solvent mix, preferably a mix of dichloromethane and methanol in weight ratio of 80:20 to 95:5, the biodegradable polymer in a concentration of from 10 w/w % to 30 w/w %, and the somatostatin analogue or pharmaceutically acceptable salt thereof in a concentration adequate to achieve a predetermined drug loading i.e., 10 w/w % to 15 w/w %,
ii. Preparing an aqueous phase comprising 0.1 w/v % to 10 w/v % e.g., 0.5 w/v % to 5 w/v % e.g., 1 w/v % of hydrophilic stabiliser, preferably PVA, and 2 w/w % to 5 w/w % of a salting-out agent, preferably sodium chloride,
iii. Continuously mixing the organic and aqueous phases of steps i. and ii. in a volume ratio of 1:80 to 1:250, to form an emulsion,
iv. Removing the organic solvents by solvent evaporation or solvent extraction from the emulsion of step iii,
v. Drying the biodegradable polymer microparticles obtained in step iv and sieving them through an appropriately sized sieve,
vi. Optionally repeating step v until any residual organic solvent is below a predetermined level e.g., DCM is below a level of 8000 ppm, and preferably below a level of 7500 ppm.

Wherein, with the exception of the drug loading, all w/w % and w/v % in the above process are based on the weight or volume of the respective solution.

The organic solvent mix comprises an organic solvent that is suitable for dissolving the biodegradable polymer and an organic solvent that is suitable for dissolving the somatostatin analogue or pharmaceutically acceptable salt thereof. The organic solvent is a mix of 2 or more different solvents, preferably 2 different solvents.

Non limiting examples of the organic solvent that may be used to dissolve the biodegradable polymer include ethyl acetate, acetone, dimethylformamide (DMF) tetrahydrofuran (THF), acetonitrile, or halogenated hydrocarbons, e.g., dichloromethane (DCM), chloroform or hexafluoroisopropanol (HFIP).

Non limiting examples of the organic solvent used to dissolve the somatostatin analogue or pharmaceutically acceptable salt thereof may be methyl-2-pyrolidone, dimethyl sulfoxide (DMSO), dimethylacetamide, ethanol, DMF, isopropanol, ethyl acetate, acetone, methanol, THF, acetonitrile, or halogenated hydrocarbons, e.g., methylene chloride, DCM, chloroform or HFIP.

A preferred organic solvent mix is a mix of dichloromethane (DCM) and methanol. The DCM and methanol may be mixed in a ratio of 80:20 to 95:5 e.g., 85:15, 90:10.

The biodegradable polymer, may be dissolved in the organic solvent used for dissolving it in a concentration of 10 w/w %-30 w/w % e.g., 11 w/w %, 12 w/w %, 13 w/w %, 14 w/w %, 25 w/w %, 16 w/w %, 17 w/w %, 18 w/w %, 19 w/w %, 20 w/w %, 21 w/w %, 22 w/w %, 23 w/w %, 24 w/w %, 25 w/w %, 26 w/w %, 27 w/w %, 28 w/w %, 29 w/w %. Wherein all w/w % are based on the weight of the final solution prepared in step i (organic phase) of the process set out above.

The somatostatin analogue, or pharmaceutical salt thereof, may be dissolved in the organic solvent used for dissolving it in a concentration adequate to achieve a predetermined drug loading i.e, 10-15 w/w % e.g., 11 w/w %, 11.5 w/w %, 12 w/w %, 12.5 w/w %, 13 w/w %, 13.5 w/w %, 14 w/w %, 14.5 w/w %. As the skilled person would understand, to achieve a desired drug loading it may be necessary to dissolve 10% to 35% e.g., 10% to 25% e.g., 20% to 25% more of the somatostatin analogue or pharmaceutical acceptable salt thereof, e.g., for a desired drug loading of 12 w/w % it may be necessary to used dissolve 13 w/w % to 15 w/w % of the somatostatin analogue or pharmaceutically acceptable salt thereof, in the relevant organic solvent e.g., 11 w/w %, 11.5 w/w %, 12 w/w %, 12.5 w/w %, 13 w/w %, 13.5 w/w %, 14 w/w %, 14.5 w/w %. Wherein all w/w % are based on the weight of the final solution prepared in step i (organic phase) of the process set out above.

The term “stabiliser” as used herein refers to an ingredient that can act as an emulsifier and stabilise an emulsion i.e., the emulsion formed when the organic and aqueous phases of steps i and ii in the process set out above are mixed.

Non limiting examples of stabilisers include polyvinyl alcohol, polyvinyl pyrrolidone, carboxymethyl cellulose sodium, dextrin, polyethylene glycol, poloxamers, poly(oxyethylene)-sorbitan-fatty acid ester, sorbitan fatty acid, lecithins, and mixtures thereof. In an embodiment the stabiliser is polyvinyl alcohol (PVA).

The aqueous phase prepared in step ii of the process set out above may comprise the stabiliser in a concentration of 0.1 w/v % to 10 w/v % e.g., 0.5 w/v % to 5 w/v % e.g., 1 w/v %, 1.5 w/v %, 2 w/v %, 2.5 w/v %, 3 w/v %, 3.5 w/v %, 4 w/v %, 4.5 w/v %. Wherein all w/v % are based on the weight of the final solution prepared in step ii (aqueous phase) of the process set out above.

The term “salting-out agent” as used herein refers to an ingredient that attracts water molecules and thereby decreases the number of water molecules available to interact with the PLGA and/or somatostatin analogue or salt thereof.

The salting-out agent in step ii of the process may be a salt. Non-limiting examples of suitable salts include sodium chloride (NaCl), potassium chloride (KCl). In an embodiment of the invention the salting out agent in step ii is NaCl.

The aqueous phase prepared in step ii of the process set out above may comprise the salting-out agent in a concentration of 2 w/v % to 5 w/v %, e.g., 3 w/v % to 4 w/v % e.g., 3.1 w/v %, 3.2 w/v %, 3.3 w/v %, 3.4 w/v %, 3.5 w/v %, 3.6 w/v %, 3.7 w/v %, 3.8 w/v %, 3.9 w/v %. Wherein all w/v % are based on the weight of the final solution prepared in step ii (aqueous phase) of the process set out above.

The organic and aqueous phases prepared in steps i and ii of the process set out herein are continuously mixed in step iii i.e., the organic and aqueous phases are simultaneously fed/pumped into and mixed in a mixing apparatus e.g., a rotary mixer.

The organic solvents may be removed by solvent evaporation or solvent extraction from the emulsion. The microparticles may then be dried e.g., by freeze drying or drying in an oven, and sieved. The drying and sieving step may be repeated until any residual organic solvent is below a predetermined level.

The predetermined level of any residual solvent may be a level in accordance with the guidelines from the International Council for Harmonisation of Technical requirements for Pharmaceuticals or Human use ICH Q3C. In the case of DCM the predetermined level of residual solvent may be a level of 8000 ppm or below. In the case of methanol the predetermined level of residual solvent may be a level of 40,000 ppm or below e.g., 3000 ppm or below e.g., 500 ppm or below.

Sieving is done using a sieve with a mesh size that yields the particle sizes specified hereinabove and below. Preferably, the sieve is a 180 μm sieve although smaller or larger opening sizes such as 160 μm or 200 μm or any other value in the range of 150 μm to 200 μm may as well be used.

Once the biodegradable polymer e.g., PLGA, microparticles e.g., microspheres, have been obtained, they may be suspended in a non-aqueous or aqueous liquid vehicle. The biodegradable polymer e.g., PLGA, microparticles e.g., microspheres, may be suspended in the aqueous or non-aqueous liquid vehicle via simply mixing.

In another aspect of the invention there is provided the pharmaceutical composition of the invention for use as a medicament. The medicament may be for use in long term therapy e.g., long term maintenance therapy, for a disease.

In another aspect of the present invention there is provided the pharmaceutical composition of the invention for use in the prevention and/or treatment of a disease.

The pharmaceutical composition of the invention may be for use in the prevention and/or treatment of any diseases for which a somatostatin analogue, or a pharmaceutically acceptable salt thereof, has a therapeutic effect. Such diseases include, for example:
I. autosomal dominant polycystic kidney disease (ADPKD), and polycystic liver disease (PLD),
II. gigantism
III. Cushing's disease,
IV. acromegaly (wherein treatment may reduce, control, normalise, and/or maintain over the long-term (long-term maintenance therapy), GH and/or IGF-1 blood levels) e.g., in patients where the condition is inadequately controlled by surgery or radiotherapy, in patients unfit or unwilling to undergo surgery or radiotherapy, or in irradiated patients, until radiotherapy is effective,
V. TSH-secreting pituitary adenomas (which may be benign) e.g., in patients when secretion has not normalized after surgery and/or radiotherapy, in patients unfit or unwilling to undergo surgery or radiotherapy, or in irradiated patients, until radiotherapy is effective.
VI. vasoactive intestinal peptide tumours (VIPomas) e.g., where it may treat the watery diarrhea (which may be severe) associated with this disease, treatment may be before or after surgery, or in patients unfit or unwilling to undergo surgery or radiotherapy, or in irradiated patients until radiotherapy is effective,
VII. neuroendocrine neoplasms (NENs) e.g., neuroendocrine tumours (NETs) (which may be advanced) e.g., gastro-entero-pancreatic neuroendocrine tumours GEP-NETs e.g., advanced NETs of the midgut or of unknown primary origin where non-midgut sites of origin have been excluded, treatment may be before or after surgery, or in patients unfit or unwilling to undergo surgery or radiotherapy, radioligand therapy, or concomitants to radiotherapy, radioligand therapy,
VIII. carcinoid syndrome associated with neuroendocrine tumours, such as functional gastro-entero-pancreatic endocrine tumours, e.g., where treatment may suppress or inhibit the diarrhoea (which may be severe) and/or flushing episodes associated with this disease, treatment may be before or after surgery, or in patients unfit or unwilling to undergo surgery or radiotherapy, radioligand therapy, or concomitants to radiotherapy, radioligand therapy,
IX. prevention of complications following pancreatic surgery
X. emergency management to stop bleeding and to protect from rebleeding owing to gastroesophageal varices in patients with cirrhosis.

In an embodiment the pharmaceutical composition of the invention is for use in the prevention and/or treatment of a disease in which a somatostatin analogue, or a pharmaceutically acceptable salt thereof, has a therapeutic effect, and wherein the disease is preferably selected from the group consisting of autosomal dominant polycystic kidney disease, Cushing's disease, polycystic liver disease, acromegaly, gigantism, TSH-secreting pituitary adenomas, carcinoid syndrome, vasoactive intestinal peptide tumours, neuroendocrine neoplasms including neuroendocrine tumours including gastro-entero-pancreatic neuroendocrine tumours.

In a more specific embodiment, the disease is selected from the group consisting of acromegaly and gastro-entero-pancreatic neuroendocrine tumours (GEP-NETs).

In another aspect of the present invention there is provided the use of a pharmaceutical composition of the invention for use in the manufacture of a medicament for use in the treatment of a disease, and preferably a disease in which a somatostatin analogue, or a pharmaceutically acceptable salt thereof, has a therapeutic effect e.g., acromegaly and gastro-entero-pancreatic neuroendocrine tumours (GEP-NETs).

In another aspect of the present invention there is provided the use of a pharmaceutical composition of the invention for use in a method of treating a disease, wherein the method comprises the step of administering a composition of the invention to a patient in need thereof, and preferably a disease in which a somatostatin analogue, or a pharmaceutically acceptable salt thereof, has a therapeutic effect e.g., acromegaly and gastro-entero-pancreatic neuroendocrine tumours (GEP-NETs).

Of course, this means that the present invention also provides methods of treating any of the medical indications specified herein, comprising the step of administering the composition of the invention to a patient in need thereof.

The pharmaceutical composition of the invention may be administered to a patient parentally e.g., via an injection, or via surgical implantation.

In an embodiment, the pharmaceutical composition of the invention is administered to a patient parentally. In a more specific embodiment, said composition of the invention is administered to a patient via intramuscular or subcutaneous injection. The subcutaneous injection may be deep subcutaneous. Preferably the composition of the invention is administered to a patient via intramuscular injection.

The pharmaceutical composition of the invention may be administered as often as necessary e.g., depending on the sustained release period of said composition. Said composition may, for example be administered every month (about every 30 days e.g., 28 to 30 days), every 2 months (about every 60 days e.g., 56-60 days), every 3 months (about 90 days e.g., 84 to 90 days), or every 4 months (about 120 days e.g., 112 to 120 days), or any time period in-between any of these values.

In an embodiment, the pharmaceutical composition of the invention is a pharmaceutical composition that provides a sustained release of the somatostatin analogue, or a pharmaceutically acceptable salt thereof, over 30 days or more and is administered once every month e.g., about every 30 days e.g., every 20-35 days e.g., 21 days, 22 days, 23 days, 24 days, 25 days, 26 days, 27 days, 28 days, 29 days, 30 days, 31 days, 32 days, 33 days, 34 days.

In an embodiment, the pharmaceutical composition of the invention is a sustained release pharmaceutical composition that provides a sustained release of the somatostatin analogue, or a pharmaceutically acceptable salt thereof, over 60 days or more and is administered once every 2 months e.g., about every 60 days e.g., every 50-65 days e.g., 51 days, 52 days, 53 days, 54 days, 55 days, 56 days, 57 days, 58 days, 59 days, 60 days, 61 days, 62 days, 63 days, 64 days.

In an embodiment, the pharmaceutical composition of the invention is a sustained release pharmaceutical composition that provides a sustained release of the somatostatin analogue, or a pharmaceutically acceptable salt thereof, over 84 days or more e.g., 84 days, and is administered once every 3 months e.g., every 84 to 90 days e.g., every 84 days.

In a more specific embodiment, the pharmaceutical composition of the invention is a 3 month sustained release pharmaceutical composition that provides a sustained release of the somatostatin analogue, or a pharmaceutically acceptable salt thereof, over 84 days or more e.g., 84 days, and is administered once every 3 months e.g., every 84 days.

In another embodiment, the pharmaceutical composition of the invention is a sustained release pharmaceutical composition that provides a sustained release of the somatostatin analogue, or a pharmaceutically acceptable salt thereof, over 90 days or more and is administered once every 3 months e.g., about every 90 days e.g., every 80-95 days e.g., 81 days, 82, days, 83 days, 84 days, 85 days, 86 days, 87 days, 88, days 89 days, 98 days, 91 days, 92 days, 93 days, 94 days.

In yet another embodiment, the pharmaceutical composition of the invention is a sustained release pharmaceutical composition that provides a sustained release of the somatostatin analogue, or a pharmaceutically acceptable salt thereof, over 120 days or more and is administered once every 4 months e.g., about every 120 days e.g., every 110-125 days e.g., 111 days, 112 days, 113 days, 114 days, 115 days, 116 days, 117 days, 118 days, 119 days, 120 days, 121 days, 122 days, 123 days, 124 days.

The pharmaceutical composition of the invention may be administered in any effective dose.

The term “effective dose” as used herein refers to a dose that results in a sustained release (i.e., release of an effective amount of the somatostatin analogue, or pharmaceutically acceptable salt thereof, over the predetermined sustained release period e.g., 30 days or more, 60 days or more, 90 days or more, or 120 days or more). It is understood that the exact dose will depend on a number of factors, including the specific somatostatin analogue in question, the condition to be treated, the predetermined sustained release period e.g., 30 days or more, 60 days or more, 90 day or more, or 120 days or more. The effective dose may also depend on specific patient characteristics and/or their response to the treatment. It is well within the purview of the skilled person to determine an effective dose e.g., balancing both efficacy and side effects/adverse reactions.

If the somatostatin analogue is octreotide, or a pharmaceutically acceptable salt thereof, particularly effective doses of the pharmaceutical composition of the invention may be doses corresponding to a dose of the octreotide, of 10 mg to 240 mg e.g., 10 mg to 180 mg, for example 20 mg to 180 mg, 25 mg, to 120 mg, 30 mg to 120 mg, 25 mg to 90 mg, or 30 mg to 90 mg e.g., 30 mg, 35 mg, 40 mg, 45 mg, 50 mg, 55 mg, 60 mg, 65 mg, 70 mg, 75 mg, 80 mg, 85 mg, or 90 mg, or the equivalent dose of a pharmaceutically acceptable salt thereof.

These doses may be particularly effective for the treatment of acromegaly and/or GEP-NET in a patient.

Since the composition of the present invention may release a somatostatin analogue or pharmaceutically acceptable salt thereof over 30 days or more, as specified in more detail hereinabove and below, a single administration of the composition of the present invention can be sufficient to accomplish therapeutically effective plasma levels over such a prolonged period of time. This means, in turn, that each dosage indication provided herein is at the same time an indication of a suitable unit dose that may for instance be contained in a prefilled syringe.

For a pharmaceutically acceptable salt of a somatostatin analogue e.g., octreotide acetate, the equivalent dose is calculated as the weight of the pharmaceutically acceptable salt of a somatostatin analogue being provided in the same molar amount as the molar amount of the free base e.g., octreotide, being provided in the dose specified herein. That is, equivalency of doses of a free base drug and its salts is determined on the basis of the molar amounts being the same.

In an embodiment of the invention the pharmaceutical composition of the invention provides a sustained release of the somatostatin analogue octreotide over 30 days or more and is administered about every 30 days (e.g., 1 month) in a dose corresponding to a dose of octreotide of 10 to 180 mg e.g., 25 mg to 120 mg, 25 mg to 105 mg e.g., 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg, or an equivalent dose of a pharmaceutically acceptable salt thereof. A particularly effective dose may be 25 mg to 105 mg, or an equivalent dose of a pharmaceutically acceptable salt thereof. Preferably administration is for the treatment of acromegaly and/or GEP-NET in a patient

In an embodiment of the invention the pharmaceutical composition of the invention provides a sustained release of the somatostatin analogue octreotide over 60 days or more and is administered once about every 60 days (e.g., 2 months) in a dose corresponding to a dose of octreotide of 10 to 180 mg e.g., 25 mg to 120 mg, 25 mg to 105 mg e.g., 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, or an equivalent dose of a pharmaceutically acceptable salt thereof. A particularly effective dose may be 25 mg to 105 mg, or an equivalent dose of a pharmaceutically acceptable salt thereof. Preferably administration is for the treatment of acromegaly and/or GEP-NET in a patient

In an embodiment of the invention the pharmaceutical composition of the invention provides a sustained release of the somatostatin analogue octreotide, or a pharmaceutically acceptable salt thereof, over 84 days or more e.g., 84 days, and is administered once every 84 days (i.e., about 3 months) in a dose corresponding to a dose of octreotide, of 10 to 180 mg e.g., 25 mg to 120 mg, 25 mg to 105 mg e.g., 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, or an equivalent dose of a pharmaceutically acceptable salt thereof. A particularly effective dose in this embodiment may be 25 mg to 105 mg e.g., 60 mg, 90 mg, or 120 mg, or an equivalent dose of a pharmaceutically acceptable salt thereof. Preferably, according to this embodiment, administration is for the treatment of acromegaly and/or GEP-NET in a patient.

In another embodiment of the invention the pharmaceutical composition of the invention provides a sustained release of the somatostatin analogue octreotide over 90 days or more and is administered once about every 90 days (e.g., 3 months) in a dose corresponding to a dose of octreotide of 10 to 180 mg e.g., 25 mg to 120 mg, 25 mg to 105 mg e.g., 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, or an equivalent dose of a pharmaceutically acceptable salt thereof. A particularly effective dose may be 25 mg to 105 mg, or an equivalent dose of a pharmaceutically acceptable salt thereof. Preferably administration is for the treatment of acromegaly and/or GEP-NET in a patient

In yet another embodiment of the invention the pharmaceutical composition of the invention provides a sustained release of the somatostatin analogue octreotide over 120 days or more and is administered once about every 120 days (e.g., 4 months) in a dose corresponding to a dose of octreotide of 10 to 180 mg e.g., 25 mg to 120 mg, 25 mg to 105 mg e.g., 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, or an equivalent dose of a pharmaceutically acceptable salt thereof. A particularly effective dose may be 25 mg to 105 mg, or an equivalent dose of a pharmaceutically acceptable salt thereof. Preferably administration is for the treatment of acromegaly and/or GEP-NET in a patient

The administration of a pharmaceutical composition of the invention, in particular wherein the somatostatin analogue comprised within the biodegradable polymer microparticles is octreotide or a pharmaceutically acceptable salt thereof, may result in a suppression/reduction of serum growth hormone (GH) levels and/or serum insulin-like growth factor (IGF1) levels in a patient e.g., in a patient suffering from acromegaly.

Said suppression/reduction in serum GH levels may result in a GH serum level of 3 μg/L or less e.g., 2.5 μg/L or less e.g., 2 μg/L or less e.g., 1.5 μg/L or less, e.g., less than 1 μg/L or less.

Serum Human Growth Hormone (GH) levels may be determined using the IDS-iSYS Multi-Discipline Automated System. This assay is based on chemiluminescence technology. Samples are incubated with a biotinylated monoclonal anti-GH antibody and streptavidin labelled magnetic particles. The magnetic particles are “captured” using a magnet and a wash step performed. An acridinium labelled anti-GH monoclonal antibody is added and following a further incubation step a second wash step is performed. Trigger reagents are added, the resulting light emitted by the acridinium label is directly proportional to the concentration of GH in the original sample.

The reduction in serum IGF1 levels may be up to 50% from baseline (the IGF1 level in a patient before administration of the pharmaceutical composition of the invention) e.g., up to 40%, up to 30%, up to 20%. IGF1 levels may be measured using a validated IDS-iSYS method or a validated LC-MS/MS methods such as the method set out in Example 10B herein.

The reduction in IGF1 and/or GH serum levels may be seen over, or substantially over, a predetermined release period e.g., over a predetermined release period e.g., 84 days or more e.g., 90 days or more, minus a lag period. The lag period may for example be 7 days or less.

The administration of a composition of the invention may result in a degree of suppression/reduction of GH and/or IGF1 e.g., over 84 days, comparable to that seen following administration of currently marketed sustained release formulations e.g., Sandostatin® LAR® when administered in corresponding dosages e.g. 3× monthly 30 mg Sandostatin LAR in comparison to a composition of the invention administered as a single dose in an amount corresponding to 90 mg of octreotide.

As previously stated, the bioavailability of a somatostatin analogue e.g., octreotide, comprised within the composition of the invention may be surprisingly high in comparison to currently marketed sustained release formulations e.g., Sandostatin® LAR®. This may enable reduced doses of a somatostatin analogue to be administered whilst achieving the same effect as higher doses (possibly administered in multiple injections) of other formulations such as Sandostatin® LAR®.

The bioavailability of the somatostatin analogue e.g., octreotide, in the composition of the invention may be higher e.g., 20%, 50%, 60%, 70%, 80%, 90%, 100% higher or more e.g., 1.2, 1.3, 1.4, 1.5, 1.6, 1.7, 1.8, 1.9, 2, 3 or more times higher, more bioavailable than in other formulations such as Sandostatin® LAR®, and may therefore enable administration of lower doses of the somatostatin analogue e.g., octreotide, to treat a condition.

In another aspect of the present invention there is provided a kit comprising:
i. a pharmaceutical composition of the invention,
ii. optionally a liquid vehicle for suspension, and
iii. a vial or a syringe optionally prefilled with the pharmaceutical composition of item (i).

The pharmaceutical composition of the invention of item i, either in the form of dried biodegradable microparticles or in the form of a suspension in a liquid vehicle may be comprised without the liquid vehicle for suspension of item ii in the vial or prefilled syringe of item iii. The liquid vehicle of item ii may be provided in a separate vial or compartment of a syringe together with the pharmaceutical composition of the invention of item i, being in the form of dried biodegradable microparticles and also being provided in a separate vial or compartment of said syringe. The pharmaceutical composition of the invention of item i, e.g., composition of dried biodegradable polymer microparticles, may be suspended, via simple mixing, in the vehicle for suspension of item ii, e.g., water (aqueous) vehicle containing pharmaceutical excipients, prior to injection into a patient.

Alternatively, the pharmaceutical composition of the invention e.g., composition of dried biodegradable polymer microparticles of item i, may be separated from a vehicle for suspension according to item ii in a double chamber pre-filled syringe.

In another alternative, the pharmaceutical composition may be a liquid suspension, comprising biodegradable polymer microparticles, that is ready for injection, e.g., a suspension comprising the biodegradable polymer microparticles in a non-aqueous vehicle, e.g., MCT oil. Said liquid suspension may be provided in a vial or prefilled syringe.

Any embodiment disclosed herein can be combined with any other embodiment disclosed herein unless explicitly stated otherwise.

As used in this disclosure and the appended claims, the singular forms “a,” “an” and “the” include plural referents unless the context clearly dictates otherwise. Thus, for example, reference to “a material” or “the material” includes two or more materials. The words “comprise,” “comprises” and “comprising” are to be interpreted inclusively rather than exclusively. Likewise, the terms “include,” “including” and “or” should all be construed to be inclusive, unless such a construction is clearly prohibited from the context. However, the compositions disclosed herein may lack any element that is not specifically disclosed. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive.

All percentages and ppm values expressed herein are by weight of the total weight of the composition unless expressed otherwise.

As used herein, “about” and “approximately” are understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of 1 to 10 should be construed as supporting a range of 1 to 8, 3 to 7, 1 to 9, 3.6 to 4.6, 3.5 to 9.9, and so forth.

While the invention has been illustrated and described with respect to illustrative embodiments and modes of practice, it will be apparent to those skilled in the art that various modifications and improvements may be made without departing from the scope and spirit of the invention. Accordingly, the invention is not to be limited by the illustrative embodiments and modes of practice.

There now follows a series of non-limiting examples that serve to illustrate the invention.

### EXAMPLES

### Example 1 Microsphere Preparation

The following method was used to make the microspheres (compositions A, B, C, and D) listed in Table I.

1. An appropriate amount of dichloromethane and methanol was mixed in a glass container to obtain a solution with the appropriate concentration of methanol as stated in Table I (column “methanol conc.”). An appropriate amount of PLGA polymer was then dissolved in this solution to give a polymer concentration as stated in Table I (column “PLGA conc).”. Following this, an appropriate amount of octreotide acetate was weighed and added to this solution under magnetic stirring, (the octreotide acetate was added in an amount that equated to a theoretical drug load for the later formed microspheres as stated in Table I (column “theoretical drug load”). The solution obtain is referred to as the “organic solution”.
2. An appropriate amount of PVA 4-88 was dissolved in an appropriate amount of deionized water to form a 1% PVA 4-88 solution. An appropriate amount of NaCl was then dissolved in this 1% PVA 4-88 solution to obtain a solution” with a concentration of NaCl as stated in Table I (column “NaCl conc.”). The solution obtained is referred to as the “aqueous solution”.
3. The organic solution was mixed with the aqueous solution by pumping the organic solution, with a flexible tube pump at a rate of 5 mL/min, into a rotor-stator equipment and by pumping the aqueous solution, using a peristaltic pump at a rate of 650 mL/min, into the same rotor-stator equipment. The two solutions were mixed in the rotor-stator chamber at 5000 rpm speed. The microsphere suspension obtained by O/W emulsion was collected in a glass beaker.
4. The microspheres were collected by filtration (5 um) and then washed several times (to remove any octreotide acetate and/or PVA on their surface) with an appropriate amount of a solution of 10% methanol in water. Following this the microspheres were dried overnight (16 h) at 30° C. under vacuum. The dried microspheres were sieved through 180 um sieve and added, under azote, into glass vials. The microspheres were then sterilized by X-ray irradiation with a dose of 25 kGy.
5. The drug load was quantified via liquid chromatography. The drug loads are shown in table I column named “drug load real”.

### Example 2: In Vitro Characterization of Burst

For each of the compositions listed in table I (A, B, C and D), the percentage of dissolution after 5 h was determined at 37° C. in 900 mL of pH4 acetate buffer using a USP2 paddle apparatus. After introduction in the dissolution medium of a composition amount equivalent to 30, 60 or 90 mg of drug substance, the paddle rotation was started at a speed of 75 rpm. After 5 h, the dissolution medium was sampled, filtrated, and the drug dissolved in the filtrate was quantified using a suitable high-performance liquid chromatography method. Results are shown in table I.

### Example 3: Determination of Specific Surface Area and Particle Size

The specific surface area of the microspheres of composition A, B, C, and D was determined by measuring the nitrogen adsorption isotherm at 77 K on appropriately degassed samples of more than 0.3 g, followed by application of the Brunauer, Emmett and Teller model in the 0.05 to 0.2 relative pressure range of the isotherm, this range comprising at least 7 data points.

Specific surface area (SSA) was measured using multipoint BET surface area by Nitrogen adsorption (volumetric technique using Micromeritics TriStar II 3020). Approximately 350 mg of each sample was added to a sample tube, before being put under vacuum for 1 hour at 20° C. After conditioning, the net mass of each sample was rerecorded, and these values were used for the analysis. During analysis, the sample tubes were surrounded by an isothermal jacket and contained a filler rod. The BET surface area was measured using the following parameters:
Analysis Adsorptive: N2
Analysis Bath Temp.: 77.300 K
Warm Free Space: Measured
Cold Free Space: Measured
Equilibration Interval: 5 s
Low Pressure Dose: None
Automatic Degas: No
Relative Pressure Tolerance: 5%
Minimum equilibration delay at P/Po>0.995: 600 seconds
Recorded P/PO range: 0.05-0.99 (88 points in 0.012 intervals)

The specific surface area results are shown in table I.

The particle size distribution was determined by wet laser diffraction using a Malvern mastersizer 3000. Each sample was resuspended in an aqueous solution of sodium carboxymethyl cellulose (1.5% w/w) and polysorbate 80 (0.1%) and then diluted/dispersed in purified water in a hydro MV dispersion unit (stirring at 2000 rpm) until reaching a stable laser obscuration of 10% to 20%. The sample was then subject to 3 mins of ultra-sonification (MV dispersion unit setting medium (50%)). The volume-weighted size distribution was calculated by means of the Mie theory using the following parameters: dispersant refractive index of 1.33; real particle refractive index of 1.52; imaginary particle refractive index 0.001.

TABLE I

| | Polymer nature | | | | | | | | | | |
|---|---|---|---|---|---|---|---|---|---|---|---|
| | (i.v. = inherent | | | | | | | | | | |
| | Viscosity (as measured | | | | | | Dv10 | Specific | In vitro | | |
| | at a concentration of | Drug load | Drug load | Methanol | PLGA | NaCl | Dv50 | Surface | release | In vivo | In vivo |
| | 0.5 w/w % in | theorical | real | conc | conc | conc | Dv90 | area | at 5 h | Cmax | Cmax SD |
| Batch | chloroform at 25° C.)) | (%) | (%) | (%) | (%) | (%) | (μm) | (m²/g) | (%) | (ng/mL) | (ng/mL) |
| Composition | PLGA 75:25 acid | 15 | 12 | 20 | Between | 5 | 55.0 | | | 48.78 | 13.36 |
| A* | iv 0.3 dL/g | | | | 10-30 | | 115 | | | | |
| | End groups COOH | | | | | | 208 | | | | |
| Composition | PLGA 85:15 acid | 14.5 | 12 | 6 | Between | 0 | 29.1 | 0.578 | 7% | 255.78 | 188.32 |
| B* | iv 0.3 dL/g | | | | 10-30 | | 74.7 | | | | |
| | End groups COOH | | | | | | 150 | | | | |
| Composition | PLGA 85:15 acid | 22 | 20 | 11 | Between | Between | 47.0 | | 28% | 438.17 | 116.02 |
| C* | iv 0.3 dL/g | | | | 10-30 | 3-4 | 94.6 | | | | |
| | End groups COOH | | | | | | 167 | | | | |
| Composition | PLGA 85:15 acid | 13 | 12 | 7 | Between | Between | 25.6 | 0.206 | <3% | 2.85 | 1.00 |
| D | iv 0.3 dL/g | | | | 10-30 | 3-4 | 63.4 | | | | |
| | End groups COOH | | | | | | 123 | | | | |

*Comparative composition

### Example 4 In Vivo Pharmacokinetic Profile of Composition D (in Rats)

The composition D microspheres were suspended in a physiologically acceptable lipophilic vehicle to obtain a drug suspension concentration of 4.8 mg/mL. The resulting suspension was subcutaneously injected, in an amount equating to a single dose of octreotide of 24 mg/kg, into 6 males Sprague Dawley rats weighing 200-250 g (5-6 weeks old) at the day of treatment. After defined time periods, plasma samples were taken and analyzed for the octreotide concentration. Results are shown in table II and FIG. 1.

TABLE II

| | Mean | |
|---|---|---|
| | plasmatic | |
| Time | concentration | SD |
| (days) | ng/mL | ng/mL |
| 0.021 | 2.85 | 1.00 |
| 0.042 | 2.33 | 0.65 |
| 0.083 | 1.34 | 0.78 |
| 0.167 | 0.68 | 0.49 |
| 0.334 | 0.46 | 0.14 |
| 1 | 0.17 | 0.07 |
| 2 | 0.24 | 0.08 |
| 3 | 0.22 | 0.07 |
| 7 | 0.29 | 0.11 |
| 14 | 0.47 | 0.17 |
| 21 | 1.21 | 0.59 |
| 28 | 1.24 | 0.57 |
| 35 | 1.13 | 0.45 |
| 42 | 2.00 | 0.45 |
| 49 | 1.93 | 0.59 |
| 56 | 2.43 | 0.75 |
| 63 | 2.47 | 0.85 |
| 70 | 2.58 | 1.00 |
| 77 | 2.48 | 1.15 |
| 84 | 2.68 | 2.11 |
| 91 | 1.59 | 0.92 |
| 98 | 2.15 | 1.30 |
| 105 | 0.98 | 0.60 |
| 112 | 1.29 | 1.23 |
| 119 | 1.11 | 0.89 |
| 126 | 0.78 | 0.54 |
| 140 | 0.36 | 0.17 |
| 154 | 0.25 | 0.12 |
| 168 | 0.17 | 0.07 |
| 182 | 0.14 | 0.07 |
| 196 | 0.10 | 0.03 |
| 210 | 0.05 | 0.02 |

### Example 5: In Vivo Pharmacokinetic Profile of Composition A (in Rats)

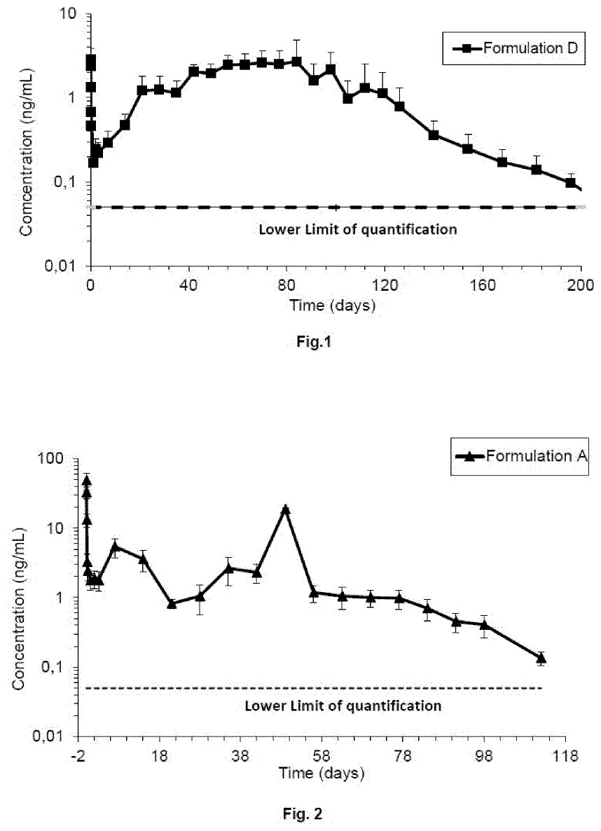

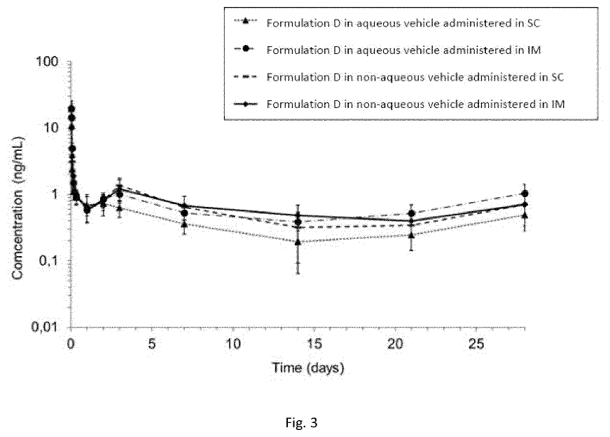

The composition A microspheres were suspended in a physiologically acceptable lipophilic vehicle to obtain a drug suspension concentration of 4.8 mg/mL. The resulting suspension was subcutaneously injected, in an amount equating to a single dose of octreotide of 24 mg/kg, into 6 males Sprague Dawley rats weighing 200-250 g (5-6 weeks old) at the day of treatment. After defined time periods, plasma samples were taken and analyzed for the octreotide concentration. Results are shown in table III and FIG. 2.

TABLE III

| | Mean plasmatic | |
|---|---|---|
| Time | concentration | SD |
| (days) | (ng/mL) | ng/mL |
| 0.021 | 48.78 | 13.36 |
| 0.042 | 32.83 | 6.11 |
| 0.083 | 13.22 | 2.84 |
| 0.167 | 3.26 | 0.97 |
| 0.333 | 2.41 | 0.67 |
| 1 | 1.76 | 0.47 |
| 2 | 1.88 | 0.54 |
| 3 | 1.80 | 0.54 |
| 7 | 5.42 | 1.66 |
| 14 | 3.58 | 1.23 |
| 21 | 0.81 | 0.12 |
| 28 | 1.05 | 0.49 |
| 35 | 2.63 | 1.16 |
| 42 | 2.31 | 0.72 |
| 49 | 18.74 | 0.81 |
| 56 | 1.18 | 0.33 |
| 63 | 1.05 | 0.37 |
| 70 | 1.01 | 0.28 |
| 77 | 0.99 | 0.31 |
| 84 | 0.70 | 0.23 |
| 91 | 0.45 | 0.14 |
| 98 | 0.41 | 0.14 |
| 112 | 0.14 | 0.03 |

### Example 6a: Sustained Release Composition for Injection (Single Dose)

Composition D microspheres (in an amount corresponding to 30 mg of octreotide) are aseptically filled into a two-chamber syringe (TCS) consisting of one compartment containing the microparticles, and one compartment containing 2.4 mL of an aqueous vehicle for suspension of the microparticles. The aqueous vehicle being 4.25% mannitol, 0.1% tween and 1.5% Sodium carboxymethyl cellulose in water.

The microparticles and the aqueous vehicle are mixed just prior to injection.

The composition may provide a sustained release of octreotide for 1 month (30 days), 2 months (60 days), 3 months (90 days), or 4 months (120 days). The sustained release period can be 1, 2, 3 or 4 months dependent on the characteristics of the patients and their response to the therapy

### Example 6b: Sustained Release Composition for Injection (Single Dose)

Composition D microspheres (in an amount corresponding to 30 mg of octreotide) are aseptically filled into a two-chamber syringe (TCS) consisting of one compartment containing the microparticles, and one compartment containing 2.4 mL of a non-aqueous liquid for suspension of the microparticles. The non-aqueous vehicle being MCT oil (MIGLYOL® 812).

The microparticles and the non-aqueous vehicle are mixed just prior to injection.

The composition may provide a sustained release of octreotide for 1 month (30 days), 2 months (60 days), 3 months (90 days), or 4 months (120 days). The sustained release period can be 1, 2, 3 or 4 months dependent on the characteristics of the patients and their response to the therapy.

### Example 7a: Sustained Release Composition for Injection (Single Dose)

Composition D microspheres (in an amount corresponding to 60 mg of octreotide) are aseptically filled into a two-chamber syringe (TCS) consisting of one compartment containing the microparticles, and one compartment containing 2.4 mL of an aqueous vehicle for suspension of the microparticles. The aqueous vehicle being 4.25% mannitol, 0.1% tween and 1.5% Sodium carboxymethyl cellulose in water.

The microparticles and the aqueous vehicle are mixed just prior to injection.

The composition may provide a sustained release of octreotide for 1 month (30 days), 2 months (60 days), 3 months (90 days), or 4 months (120 days). The sustained release period can be 1, 2, 3 or 4 months dependent on the characteristics of the patients and their response to the therapy.

### Example 7b: Sustained Release Composition for Injection (Single Dose)

Composition D microspheres (in an amount corresponding to 60 mg of octreotide) are aseptically filled into a two-chamber syringe (TCS) consisting of one compartment containing the microparticles, and one compartment containing 2.4 mL of a non-aqueous liquid for suspension of the microparticles. The non-aqueous vehicle being MCT oil (MIGLYOL® 812).

The microparticles and the non-aqueous vehicle are mixed just prior to injection.

The composition may provide a sustained release of octreotide for 1 month (30 days), 2 months (60 days), 3 months (90 days), or 4 months (120 days). The sustained release period can be 1, 2, 3 or 4 months dependent on the characteristics of the patients and their response to the therapy.

### Example 8a: Sustained Release Composition for Injection (Single Dose)

Composition D microspheres (in an amount corresponding to 90 mg of octreotide) are aseptically filled into a two-chamber syringe (TCS) consisting of one compartment containing the microparticles, and one compartment containing 2.4 mL of an aqueous vehicle for suspension of the microparticles. The aqueous vehicle being 4.25% mannitol, 0.1% tween and 1.5% Sodium carboxymethyl cellulose in water.

The microparticles and the aqueous vehicle are mixed just prior to injection.

The composition may provide a sustained release of octreotide for 1 month (30 days), 2 months (60 days), 3 months (90 days), or 4 months (120 days). The sustained release period can be 1, 2, 3 or 4 months dependent on the characteristics of the patients and their response to the therapy.

### Example 8b: Sustained Release Composition for Injection (Single Dose)

Composition D microspheres (in an amount corresponding to 90 mg of octreotide) are aseptically filled into a two-chamber syringe (TCS) consisting of one compartment containing the microparticles, and one compartment containing 2.4 mL of a non-aqueous liquid for suspension of the microparticles. The non-aqueous vehicle being MCT oil (MIGLYOL® 812).

The microparticles and the non-aqueous vehicle are mixed just prior to injection.

The composition may provide a sustained release of octreotide for 1 month (30 days), 2 months (60 days), 3 months (90 days), or 4 months (120 days). The sustained release period can be 1, 2, 3 or 4 months dependent on the characteristics of the patients and their response to the therapy.

### Example 9: Impact of the Route of Administration and Vehicle

The composition D microspheres were suspended in a physiologically acceptable aqueous or non-aqueous vehicle to obtain a drug suspension concentration of 4.8 mg/mL. The resulting suspension was subcutaneously or intramuscularly injected, in an amount equating to a single dose of octreotide of 24 mg/kg, into 6 males Sprague Dawley rats weighing 200-250 g (5-6 weeks old) at the day of treatment. After defined time periods, plasma samples were taken and analyzed for the octreotide concentration over 28 days. Results are shown in FIG. 3, where no significant difference is observed.

### Example 10A: Bioavailability

A) Sandostatin immediate release (sandostatin IR) was administered, via sub-cutaneous injection, to 15 healthy subjects in an amount equating to 0.2 mg of octreotide. Following administration, octreotide plasma concentrations were determined over the investigation period. AUCₗₐₛₜ (up to about 24 h) was calculated for each subject and normalized by the dose.

Subsequently (at least 1 week later), to the same 15 subjects, an MCT oil (MIGLYOL® 812) suspension of the composition D microspheres was administered via intramuscular injection in an amount equating to 30 mg of octreotide. Following administration, octreotide plasma concentrations were determined over the investigation period. AUCₗₐₛₜ (up to 84 to 112 days) was calculated for each subject and normalized by the dose.

The 2 AUCₗₐₛₜ normalized by the dose values were then compared intra-individually. The goal was to determine within the same subject the relative bioavailability of the composition of D microspheres compared to sandostatin IR.

For each subject the following ratio was determined:

$$\frac{Composition D microparticles AUClast / Dose ( 30 mg )}{Sandostatin IR AUClast / Dose ( 0.2 mg )}$$

where composition D microparticles AUCₗₐₛₜ corresponds to the area under the curve from Day 0, day of first administration of the composition D microparticles, up to last day the plasma octreotide concentration was quantifiable, and wherein Sandostatin IR AUCₗₐₛₜ corresponds to the area under the curve from administration of Sandostatin immediate release up to the last time the plasma octreotide concentration was quantifiable, which corresponds to about 24 h post dose.

The mean of the determined ratios was then calculated.

B) A control arm of 15 healthy subjects was administered, via sub-cutaneous injection, a dose of Sandostatin IR in an amount equating to 0.2 mg of octreotide. Following administration, octreotide plasma concentrations were determined over the investigation period. AUCₗₐₛₜ (up to about 24 h) was calculated for each subject and normalized by the dose.

Subsequently (at least 1 week later), a dose of 30 mg Sandostatin long acting (Sandostatin LAR®) was administered, via intramuscular injection, to the same 15 control arm subjects and repeated every 4 weeks for 12 weeks (3×30 mg dose resulting in a cumulative dose over the investigation period of 90 mg). AUCₗₐₛₜ over the investigation period (up to 21 days for one subject who withdrew consent or up to 70 to 84 days) was calculated for each subject using plasma octreotide concentrations, and was normalised by the cumulative dose of Sandostatin LAR given over the investigation period i.e., divided by 30 mg or 90 mg depending on what the cumulative dose was.

The 2 AUCₗₐₛₜ normalized by the dose values were then compared intra-individually for the control arm subjects. The goal was to determine within the same subject the relative bioavailability of sandostatin LAR compared to sandostatin IR.

Using data generated from the above study, the relative bioavailability of the composition D microparticles could be compared to the relative bioavailability of sandostatin LAR:

For each subject the following ratio was determined:

$$\frac{Sandostatin LAR AUClast / Dose ( 3 * 30 mg )}{Sandostatin IR AUClast / Dose ( 0.2 mg )}$$

where Sandostatin LAR AUCₗₐₛₜ corresponds to the area under the curve from Day 0, day of first administration of the Sandostatin LAR, up to last day that the plasma octreotide concentration was quantifiable, and wherein Sandostatin IR AUCₗₐₛₜ corresponds to the area under the curve from administration of Sandostatin immediate release up to last time the plasma octreotide concentration was quantifiable measured, which corresponds to about 24 h post dose.

The mean of the determined ratios was then calculated.

The mean ratio calculated in A) above was then compared to the mean calculated in B) above.

Results are presented in table IV and show that the composition D microparticles have a bioavailability equivalent to about two-fold the bioavailability of Sandostatin LAR in humans.

TABLE IV

| | | | Ratio bioavailability |
|---|---|---|---|
| | | Mean relative | of composition D |
| | Intraindividual | bio-availabilities | microparticles vs |
| N | comparison | (%) | Sandostatin LAR |
| 15 | Composition D | 22.96 | 1.8 |
| | Microparticles Versus | | |
| | Sandostatin IR | | |
| 15 | Sandostatin LAR Versus | 12.96 | |
| | Sandostatin IR | | |

All treatments were well tolerated and there were no significant differences in pattern or severity of adverse events.

### Example 10B: Bioavailability and Changes in Serum IGF1

A) IGF1 levels were measured in 7 healthy volunteer subjects. Following IGF1 measurement, sandostatin immediate release (sandostatin IR) was administered, via sub-cutaneous injection, to the same 7 healthy subjects in an amount equating to 0.2 mg of octreotide.
I. octreotide plasma concentrations were determined over the investigation period and the AUCₗₐₛₜ (up to about 24 h) for the octreotide plasma levels was calculated for each subject and normalized by the dose.

Subsequent to the above (at least 1 week later), to the same 7 subjects, an aqueous suspension of the composition D microspheres was administered via intramuscular injection in an amount equating to 90 mg of octreotide.
II. serum IGF1 concentration were determined on several occasions over the investigational period and % serum IGF changes were recorded,
III. octreotide plasma concentrations were determined over the investigation period (3 months (84 days)), and the AUC_{84d} for the octreotide plasma levels was calculated for each subject and normalized by the dose.

The I and III AUC normalized by the dose values were compared intra-individually. The goal was to determine within the same subject the relative bioavailability of the composition of D microspheres compared to sandostatin IR.

For each subject the following ratio was determined:

$$\frac{Composition D microparticles AUC 84 d / Dose ( 90 mg )}{Sandostatin IR AUClast / Dose ( 0.2 mg )}$$

where Composition D microparticles AUC_{84d} corresponds to the area under the curve from Day 0, day of first administration of the composition D microparticles, up to day 84 of measurable concentration, and wherein Sandostatin IR AUCₗₐₛₜ corresponds to the area under the curve from administration of Sandostatin immediate release up to the last quantifiable concentration, which corresponds to about 24 h post dose.

The mean of the determined ratios was then calculated.

B) IGF1 levels were measured in 14* healthy volunteer subjects (control arm). Following IGF1 measurement, the same 14* healthy subjects were administered, via sub-cutaneous injection, a dose of Sandostatin IR in an amount equating to 0.2 mg of octreotide.
IV. octreotide plasma concentrations were determined over the investigation period and the AUCₗₐₛₜ (up to about 24 h) was calculated for each subject and normalized by the dose.

Subsequently (at least 1 week later), a dose of 30 mg Sandostatin long acting (Sandostatin LAR®) was administered, via intramuscular injection, to the same 14 control arm subjects and repeated every 4 weeks for 12 weeks (3×30 mg dose resulting in a cumulative dose of 90 mg over the investigation period of 84 days.
V. Serum IGF1 concentrations were determined on several occasions over the investigational period and % serum IGF changes were recorded,
VI. octreotide plasma concentrations were determined over the investigated period and the AUC_{84d} for the octreotide plasma levels over the investigation period was calculated for each subject and was normalised by the cumulative dose of 90 mg of Sandostatin LAR given over the investigation period of 84 days.

The IV and VI AUC normalized by the dose values were then compared intra-individually for the control arm subjects. The goal was to determine within the same subject the relative bioavailability of sandostatin LAR compared to sandostatin IR.

For each subject the following ratio was determined:

$$\frac{Sandostatin LAR AUC 84 d / Dose ( 3 * 30 mg )}{Sandostatin IR AUClast / Dose ( 0.2 mg )}$$

where Sandostatin LAR AUC_{84d} corresponds to the area under the curve from Day 0, day of first administration of Sandostatin LAR, up to day 84, and wherein Sandostatin IR AUCₗₐₛₜ corresponds to the area under the curve from administration of Sandostatin immediate release up to the last quantifiable concentration, which corresponds to about 24 h post dose.

The mean of the determined ratios was then calculated.

Using data generated from the above, the relative bioavailability of the composition D microparticles could be compared to the relative bioavailability of sandostatin LAR: The mean ratio calculated in A) above was compared to the mean ratio calculated in B) above.

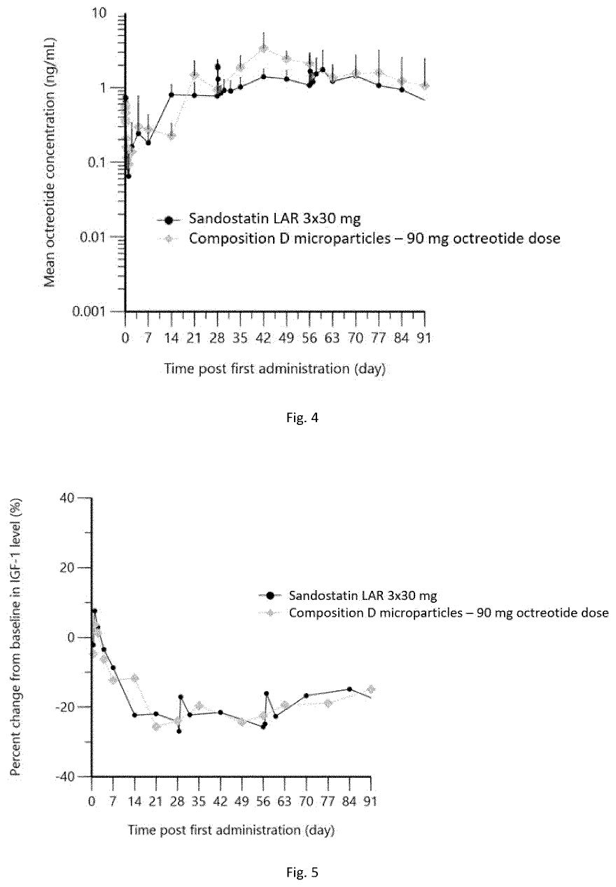

Mean plasma octreotide levels are presented in FIG. 4. Mean bioavailability results are presented in table V and show that the composition D microparticles have a bioavailability equivalent to about two-fold the bioavailability of Sandostatin LAR in humans.

TABLE V

| | | | Ratio bioavailability |
|---|---|---|---|
| | | Mean relative | of composition D |
| | Intraindividual | bio-availabilities | microparticles vs |
| N | comparison | (%) | Sandostatin LAR |
| 7 | Composition D | 27.9 | 1.7 |
| | Microparticles | | |
| | (90 mg of octreotide) | | |
| | Versus | | |
| | Sandostatin IR | | |
| 14* | Sandostatin LAR Versus | 16.1 | |
| | Sandostatin IR | | |

*Excluding subject who withdrew consent before 2^{nd} administration of Sandostatin LAR

The safety profile of the composition D microparticles (90 mg of octreotide) was consistent with the Sandostatin LAR, and there were no new safety signals.

Mean IGF1 results are presented in FIG. 5. As can be seen, administration of composition D microparticles in an amount equating to a single 90 mg dose of octreotide results in a degree of suppression of IGF1 over 84 days comparable to that seen following administration of 3× monthly 30 mg Sandostatin LAR.

In the above example (10B) IGF1 serum levels were measured using validated LC-MS/MS methods employing rabbit plasma as a surrogate matrix. The IGF-1 assay allows the quantitative determination of IGF-1 in human serum samples. Sample (using a sample volume of 100 μL of serum) processing is performed by means of protein precipitation followed by phospholipid removal using an Ostro plate from Waters Corp (more information on sample processing is given in table VI). Separation between metabolites and interfering endogenous compounds is achieved by UHPLC using a XSelect CSH C18 (100×2.1 mm, 2.5 μm) from Waters Corp. at 40° C. and using 0.1% formic acid in water as mobile phase A and acetonitrile: DMSO (90:10, v/v) as mobile phase B and operating at a gradient with an initial flow rate of 0.4 mL/min (more information on timing and flow rate and gradient is given in table VII).

A triple quad 6500 mass spectrometer equipped with a turbo ion spray source is used for detection in positive ion mode.

Quantification is based on multiple reaction monitoring (MRM) of the transitions of:
m/z 1093.6-1196.8 for IGF-1
m/z 1106.9-1211.3 for IGF-1 N15

A linear calibration curve with a 1/x² weighting factor is used ranging from 2.00 to 1000 ng/mL IGF-1 in human serum.

Additional general method directions are provided in Table VIII

TABLE VI

| □01 | Thaw the frozen rabbit plasma or human serum samples at room temperature | | | |
|---|---|---|---|---|
| | and homogenize by slowly inverting (at least 5 times), and followed by | | | |
| | vortexing for 30 seconds on a (multitube) vortexer at high rpm. | | | |
| □02 | Vortex all samples individually for 3 seconds before pipetting (at e.g. 1200 rpm | | | |
| | if a mixmate is used). Avoid the matrix touching the caps of the tubes. | | | |
| □03 | Pipette 100 μL sample in a 2.0 mL deep well plate | | | |
| | Start time pipetting | | | |
| | Reason for | □ Out of range | □ Not enough sample | □ See § 8.8 |
| | dilution (#W) | | | |
| | Dilution | Pipetted sample | Pipetted blank | LIMS ID pipette |
| | | volume (μL) | volume (μL) | (sample/blank) |
| | | | | / |

| | |
|---|---|
| | Note: #W Only applies to unknown samples. If a run contains two or more |
| | reasons for dilution, write the reason with each diluted sample on the work list |
| | Pipette 100 μL after dilution and mixing (use for further sample preparation) |
| □04 | a) Add 100 μL stock solvent (water:methanol:acetic acid (60:30:10; |
| | v/v/v) to the blanks |
| □ | b) Add 100 μL internal standard working solution (100 ng/mL) to all other |
| | wells |
| □ | c) Vortex the plate for 5 minute at 1200 rpm (thermomixer or mixmate) |
| | and room temperature |
| □05 | a) Add 400 μL ethanol to each well |
| | b) Seal the plate using a capmat (not pre-scored) and vortex the plate for |
| | 20 minutes at 200 rpm using a horizontal shaker (Gerhardt) |
| □06 | Centrifuge the plate for 5 minutes at approximately 3500 × g and 4° C. |
| □07 | Transfer 250 μL of the supernatant into a Ostro precipitation plate (using the |
| | TomTec or liquidator) TomTec method: PRA-NL-SML-2430 8 Jan. 2021 |
| | Method checked by: |
| □08 | Pipette 10.0 μL water in a 1.0 mL LoBind deep well plate |
| □09 | Elute the samples into a clean 1.0 mL LoBind deep well plate (containing |
| | 10.0 μL water) by applying 5 psi pressure for 5 minutes. If necessary |
| | apply extra pressure up to 10 psi when samples are not completely eluted |
| □10 | Evaporate the organic layer at 60° C. under a gentle stream of nitrogen for |
| | approximately 30 minutes. Do not evaporate to complete dryness!! |
| □11 | a) Add 100 μL stock solvent (water:methanol:acetic acid (60:30:10, |
| | v/v/v) to each well |
| □ | b) Cap the plate with a pre-scored silicone mat. |
| □ | c) Vortex the plate for 5 minute at 1200 rpm (thermomixer of mixmate) |
| | and room temperature |
| □12 | Centrifuge the plate for 5 minutes at approximately 3500 × g and 4° C. |

TABLE VII
Timetable

| | | | | Max. Pressure |
|---|---|---|---|---|
| Time | A | B | Flow | Limit |
| (min) | (%) | (%) | (mL/min) | (psi) |
| 0.00 | 81 | 19 | 0.400 | 18000 |
| 5.00 | 71 | 29 | 0.400 | 18000 |
| 5.10 | 50 | 50 | 0.400 | 18000 |
| 5.40 | 50 | 50 | 0.400 | 18000 |
| 5.50 | 10 | 90 | 0.400 | 18000 |
| 6.00 | 10 | 90 | 0.400 | 18000 |
| 6.10 | 81 | 19 | 0.400 | 18000 |

### 3.0 General Directions

TABLE VIII
Refer to the appropriate PRA Standard Operating Procedure for dealing with deviations from these assay
instructions.
Insulin-like growth factor 1 (IGF-1) is a hormone similar in molecular structure to insulin which plays an important
role in childhood growth, and has anabolic effects in adults. IGF-1 consists of 70 amino acids in a single chain
with three intramolecular disulfide bridges. IGF-1 has a molecular weight of 7649 Da.
Depending on age, gender and disease state, IGF-I levels in serum can range from 15-765 ng/ml. Before
preparing the validation pools, the serum must be screened and the lowest used.
The lowest screened serum is used for the preparation of QC B, QC C and QC D.
Since IGF-1 is an endogenous compound, rabbit plasma is used as a proxy matrix for the preparation of the
calibration curve and the lowest validation level
For the determination or detection of IGF-1 and its internal standard, the mass spectrometer is used in High
Mass mode.
For the determination or detection of IGF-1 and its internal standard, the 7-fold charged ion is used [M + 7].
DMSO in the mobile phase enhances the formation of the 7-fold charged ion. If tuning is required add the correct
mobile phases to optimize all parameters.
The chromatographic behavior of IGF-1 on the analytical column is sensitive to small variations in mobile
phase composition and column batches. Small changes (1%) in mobile phase composition can result in
retention shift of 0.5 up to 1 minute. For that reason the switch time of the switch valve is extended.

1. A pharmaceutical composition comprising biodegradable polymer microparticles comprising a somatostatin analogue, or a pharmaceutically acceptable salt thereof, wherein said biodegradable polymer comprises PLGA having:
a molar ratio of lactide to glycolide of 80:20 to 90:10, and
an inherent viscosity of 0.2 to 0.4 dl/g as measured at a concentration of 0.5 w/w % in chloroform at 25° C.,
and wherein said biodegradable polymer microparticles have:
a drug loading of 10 to 15 w/w %,
a Dv50 of 30 mm to 90 mm, and
a specific surface area of less than 0.50 m²/g, and preferably less than 0.40 m²/g, as measured by gas adsorption,
and wherein preferably said pharmaceutical composition provides a sustained release of the somatostatin analogue, or a pharmaceutically acceptable salt thereof, over 30 days or more, and more preferably over 30 days to 200 days, or over 60 days or more, and preferably 60 to 200 days, or over 90 days or more, and preferably 90 to 200 days.

2. The pharmaceutical composition according to claim 1 wherein the PLGA defined in claim 1 has a molar ratio of lactide to glycolide of 83:17 to 87:13, and an inherent viscosity of 0.25 to 0.35 dl/g as measured at a concentration of 0.5 w/w % in chloroform at 25° C.

3. The pharmaceutical composition according to claim 1 wherein the PLGA defined in any preceding claim comprises less than 0.5 w/w % of residual lactide and/or glycolide monomer.

4. The pharmaceutical composition according to claim 1 wherein the biodegradable polymer microparticles defined in any preceding claim have a drug loading of 11 to 14 w/w %, and preferably 11.5 to 12.5 w/w %.

5. The pharmaceutical composition according to claim 1 wherein the biodegradable polymer microparticles defined in any preceding claim are biodegradable polymer microspheres.

6. The pharmaceutical composition according to claim 1 wherein, the biodegradable polymer microparticles defined in any preceding claim have:
a Dv50 of 50 mm to 80 mm, preferably 60 mm to 75 mm, and/or
a specific surface area of 0.05 to 0.3 m²/g, as measured by gas adsorption.

7. The pharmaceutical composition according to claim 1 wherein the somatostatin analogue is octreotide or a pharmaceutically acceptable salt thereof, and preferably wherein the somatostatin analogue is a pharmaceutically acceptable salt of octreotide selected from the group consisting of octreotide acetate and octreotide pamoate.

8. The pharmaceutical composition according to claim 1 wherein the biodegradable polymer defined in any preceding claim comprises at least 50 w/w % of the PLGA defined in any preceding claim, preferably at least 85 w/w %, and more preferably 100 w/w %.

9. The pharmaceutical composition according to claim 1 wherein the biodegradable polymer microparticles defined in any preceding claim make up at least 50 w/w % of the total of all biodegradable polymer microparticles comprised in the pharmaceutical composition, preferably at least 75 w/w %, and more preferably at least 80 w/w %.

10. The pharmaceutical composition according to claim 1 wherein the composition is characterized by a release of the somatostatin analogue that fulfils the following criteria: release of less than 7% of the somatostatin analogue comprised therein over 5 hours, wherein said release is measured in vitro (at 37° C. in 900 mL of a pH4 100 mM acetate buffer) according to the method described in the European Pharmacopoeia 10, 2.9.3., wherein said composition is tested in an amount equating to 30 mg of the somatostatin analogue, and wherein said somatostatin analogue is preferably octreotide,
wherein the composition is preferably characterised by a release of the somatostatin analogue that fulfils the following test criteria: release of less than 3% of the somatostatin analogue comprised therein over 5 hours, wherein said release is measured in vitro (at 37° C. in 900 mL of a pH4 100 mM acetate buffer) according to the method described in the European Pharmacopoeia 10, 2.9.3., wherein said composition is tested in an amount equating to 30 mg of the somatostatin analogue, and wherein said somatostatin analogue is preferably octreotide.

11. The pharmaceutical composition according to claim 1 wherein the pharmaceutical composition is a liquid suspension comprising biodegradable polymer microparticles or is a composition of dried biodegradable polymer microparticles, wherein the pharmaceutical composition is preferably a liquid suspension comprising biodegradable polymer microparticles and wherein the biodegradable polymer microparticles are suspended in a water-vehicle or a non-aqueous vehicle, and preferably wherein the non-aqueous liquid vehicle is a pharmaceutically acceptable oil essentially consisting of one or more medium chain triglycerides, wherein the liquid more preferably comprises the microparticles in a concentration of 100 mg/mL to 500 mg/mL, particularly preferably, 100 mg/mL to 375 mg/ml, and most preferably 125 mg/mL to 375 mg/mL.

12. The pharmaceutical composition according to claim 1 wherein said pharmaceutical composition is sterilised by irradiation.

13. A pharmaceutical composition as defined in claim 1 for use as a medicament.

14. A method for treatment of a disease in a subject in which a somatostatin analogue, or a pharmaceutically acceptable salt thereof, has a therapeutic effect, and wherein preferably the disease is selected from the group consisting of autosomal dominant polycystic kidney disease, Cushing's disease, polycystic liver disease, acromegaly, gigantism, TSH-secreting pituitary adenomas, carcinoid syndrome, vasoactive intestinal peptide tumours, and neuroendocrine neoplasms including neuroendocrine tumours including gastro-entero-pancreatic neuroendocrine tumours, and preferably is selected from acromegaly and gastro-entero-pancreatic neuroendocrine tumours, comprising administration of a composition of claim 1 to the subject.

15. The method of claim 14, wherein the composition provides a sustained release of a somatostatin analogue over 60 days or more and is to be administered once about every 60 days in a dose corresponding to a dose of octreotide of 25 mg to 105 mg, or the equivalent dose of a pharmaceutically acceptable salt thereof, or wherein the composition provides a sustained release of a somatostatin analogue over 90 days or more and is to be administered once about every 90 days in a dose corresponding to a dose of octreotide of 25 mg to 105 mg, or the equivalent dose of a pharmaceutically acceptable salt thereof.

16. The method of claim 14, wherein the composition provides a sustained release of octreotide, or a pharmaceutically acceptable salt thereof, over 84 days or more and is administered once every 84 days in a dose corresponding to a dose of octreotide of 60 mg, 90 mg, or 120 mg, or the equivalent dose of a pharmaceutically acceptable salt thereof.

17. The method of claim 14, wherein the pharmaceutical composition is to be administered to a patient parenterally and preferably via intra-muscular or subcutaneous injection, wherein the subcutaneous injection is preferably deep-subcutaneous.

18. A kit comprising:
i. the pharmaceutical composition of claim 1,
ii. optionally a vehicle for reconstitution, and
iii. a vial or syringe optionally prefilled with the pharmaceutical composition of item (i).

19. A method of manufacturing a biodegradable polymer microparticle defined in claim 1, wherein said biodegradable polymer comprises at least 50 w/w %, preferably 85 ww % and most preferably 100 w/w %, of the PLGA defined in any preceding claim, and wherein said method comprises the steps of:
i. Preparing an organic phase comprising an organic solvent mix, preferably a mix of dichloromethane and methanol in a weight ratio of 80:20 to 95:5, the biodegradable polymer in a concentration of from 10 w/w % to 30 w/w %, and the somatostatin analogues, or pharmaceutically acceptable sale thereof, in a concentration adequate to achieve a predetermined drug loading of 10 w/w % to 15 w/w %,
ii. Preparing an aqueous phase containing 0.1 w/v % to 10 w/v % of a stabiliser, preferably PVA, and 2 w/w % to 5 w/w % of a salting-out agent, preferably sodium chloride,
iii. Continuously mixing the organic and aqueous phases of steps i. and ii. in a volume ratio of 1:80 to 1:250, to form an emulsion,
iv. Removing the organic solvents by solvent evaporation or solvent extraction from the emulsion of step iii,
v. Drying the biodegradable polymer microparticles obtained in step iv and sieving them through an appropriately sized sieve,
vi. optionally repeating step v until any residual organic solvent is below a predetermined level,
wherein, with the exception of the drug loading, all w/w % and w/v % in the above process are based on the weight or volume of the respective solution.
